(19) United States
(12) Patent Application Publication
(10) Pub. No.: US 2005/0272952 A1
(43) Pub. Date: Dec. 8, 2005

(54) Preparation of acrylic acid by heterogeneously catalyzed gas phase partial oxidation of at least one C3 hydrocarbon precursor compound

(75) Inventors: Ulrich Cremer, Mannheim (DE); Martin Dieterle, Mannheim (DE); Sabine Jourdan, Mannheim (DE); Jochen Petzoldt, Weisenheim am Berg (DE); Klaus Joachim Muller-Engel, Stutensee (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/107,919

(22) Filed: Apr. 18, 2005

Related U.S. Application Data
(60) Provisional application No. 60/566,398, filed on Apr. 30, 2004.

(30) Foreign Application Priority Data
Apr. 30, 2004 (DE) 102004021764.5

(51) Int. Cl. C07C 51/16

(52) U.S. Cl. 562/545

(57) ABSTRACT

A process for preparing acrylic acid by heterogeneously catalyzed partial oxidation of at least one C₃ hydrocarbon precursor compound, in which the secondary component formation is ≦1.5 mol %.

The present invention relates to a process for preparing acrylic acid by heterogeneously catalyzed gas phase partial oxidation of at least one C₃ hydrocarbon precursor compound.

Acrylic acid is an important monomer which finds use as such or in the form of its alkyl esters for obtaining, for example, polymers suitable as adhesives.

It is known that acrylic acid can be prepared by heterogeneously catalyzed gas phase partial oxidation of at least one of the two hydrocarbon precursor compounds each having three carbon atoms (referred to in this document as “C₃ hydrocarbon precursor compounds”), propene and propane.

The process for heterogeneously catalyzed gas phase partial oxidation of propene to acrylic acid proceeds in principle in two successive steps, the first step leading from propene to acrolein and the second step from acrolein to acrylic acid. Since there are catalysts which are capable of catalyzing either both or, in a tailored manner, in each case only one of the two steps, the heterogeneously catalyzed gas phase partial oxidation of propene to acrylic acid may in principle be carried out either in a single reaction stage or in two or more spatially successive reaction stages, and a particular reaction stage is characterized by its catalyst charge and the accompanying other, generally specific, reaction conditions.

A process for two-stage heterogeneously catalyzed partial gas phase oxidation of propene to acrylic acid is disclosed, for example, by DE-A 19927624, by DE-A 19948523, by WO 00/53557, by DE-A 19948248 and by WO 00/53558.

Starting from propane, the heterogeneously catalyzed partial gas phase oxidation to acrylic acid may likewise be carried out in a single reaction stage or in two or more spatially successive reaction stages. According to the teaching of DE-A 10245585 and of DE-A 10246119 among other documents, acrylic acid formation starting from propane generally proceeds obviously in three successive steps, of which the first step is normally the formation of propene. A description of a partial one-stage propane oxidation to acrylic acid is contained, for example, in the documents EP-A 608838, WO 0029106, JP-A 10-36311, DE-A 10316465, EP-A 1192987, EP-A 1193240 and DE-A 10338529. A three-stage partial propane oxidation to acrylic acid is described, for example, by WO 01/96270. Useful catalysts, both for the particular steps of the partial propene and of the partial propane oxidation, are generally multielement oxides, of which some are described in the prior art cited.

A feature common to all known processes for preparing acrylic acid by heterogeneously catalyzed gas phase partial oxidation of at least one C₃ hydrocarbon precursor compound is that, owing to numerous parallel and subsequent reactions proceeding in the course of the catalytic gas phase oxidation, and also owing to the preferably substantially inert diluent gases generally also to be used to prevent explosive gas mixtures, among other reasons, what is obtained is not pure acrylic acid but rather a reaction gas mixture which, in addition to acrylic acid and the acrolein intermediate recyclable into the partial oxidation, in some cases unconverted propene and/or propane, inert diluent gases (are substantially unconverted in the partial oxidation, i.e. they normally remain unchanged during the partial oxidation to an extent of more than 95 mol %, preferably of more than 97 mol % or more than 99 mol %), the carbon oxides CO and/or CO₂, the reaction products which are comparatively simple to remove from acrylic acid: acetic acid (may be removed, for example, by stripping from a product gas mixture absorbate) and water, and also in some cases remaining molecular oxygen as an oxidizing agent, comprises numerous other compounds which have at least one oxygen atom, at least one carbon atom and at least one hydrogen atom. These other compounds will be referred to in this document as secondary components.

When acrylic acid is obtained from the product gas mixture of a heterogeneously catalyzed partial oxidation of at least one C₃ hydrocarbon precursor compound, the acrylic acid has to be removed from the product gas mixture, which is generally effected by means of combinations of absorptive, extractive and/or distillative or rectificative separating processes (cf., for example, EP-A 854129, U.S. Pat. No. 4,317,926, DE-A 19837520, DE-A 19606877, DE-A 19501325, DE-A 10247240, DE-A 19924532, EP-A 982289, DE-A 19740253, DE-A 19740252, EP-A 695736, EP-A 982287 and EP-A 1041062).

A disadvantage of these removal processes is that they are very costly and inconvenient (energy-intensive and cost-intensive), which is why there has been no shortage of attempts to bring about optimization by skillful combination of the possible individual removal measures known per se. However, the results of these attempts are not fully satisfactory.

It is therefore an object of the present invention to provide remedy in relation to the above-described set of problems.

Detailed considerations have surprisingly led to the result that one possible route of remedy has hitherto essentially neither been taken nor considered. This route consists in modifying the process for heterogeneously catalyzed partial oxidation of at least one C₃ hydrocarbon precursor compound to the effect that the total amount of those constituents which make the removal of acrylic acid from the product gas mixture especially laborious in the product gas mixture, based on the amount of acrylic acid contained therein, is very low.

Such constituents which have been identified in-house include the secondary components already defined. These include in particular the aldehydes other than acrolein, such as formaldehyde, acetaldehyde, methacrolein, propionaldehyde, n-butyraldehyde, benzaldehyde, furfurals and crotonaldehyde, whose presence is known in particular to promote the tendency of acrylic acid to undesired free-radical polymerization in thermal separating processes to a particular degree (cf., for example, EP-A 854129). However, the secondary components also include lower alkene-/alkanecarboxylic acids other than acetic acid or their anhydrides, for example formic acid, propionic acid, methacrylic acid, crotonic acid, butyric acid and maleic acid, but also compounds such as protoanemonin, acetone and benzaldehyde.

One way of achieving the object defined in this application is therefore provided by a process for preparing acrylic acid by heterogeneously catalyzed partial oxidation of at least one C₃ hydrocarbon precursor compound, wherein the overall selectivity Sₒᵥₑ of secondary component formation is ≦1.5 mol %.

The selectivity Sᵢ of the formation of an individual secondary component i in mol % refers to one hundred times the quotient of the molar amount of the secondary component i formed divided by the molar amount of the at least one C₃ hydrocarbon precursor compound converted in the heterogeneously catalyzed partial oxidation. In this document, the overall selectivity Sₒᵥₑ of secondary component formation refers to the sum of the different individual selectivities Sᵢ over all secondary components i.

According to the invention, Sₒᵥₑ is preferably ≦1.4 mol % or ≦1.3 mol %, more preferably ≦1.2 mol % or ≦1.1 mol % or ≦1.0 mol % or ≦0.9 mol %, even more preferably ≦0.80 mol % or ≦0.70 mol % or ≦0.60 mol % or ≦0.50 mol %, and at best, Sₒᵥₑ, in accordance with the invention, is ≦0.40 mol % or ≦0.30 mol % or ≦0.20 mol % or ≦0.10 mol % or 0 mol %.

Frequently, Sₒᵥₑ in the process according to the invention will be ≧0.1 mol % and ≦1.5 mol %, or ≦0.20 mol % and ≦1.0 mol %, or ≦0.30 mol % or ≦0.40 mol % and ≦0.80 mol % or ≦0.70 mol %.

When propene is the starting material for the inventive heterogeneously catalyzed partial gas phase oxidation, the aforementioned values for Sₒᵥₑ are normally accompanied by molar propene conversions (based on single paths of the reaction gas mixture through all reaction stages) C^{pen} which are ≧95 mol % or ≧96 mol % or ≧97 mol %, preferably ≧98 mol % (based in each case on the starting amount of propene). Frequently, the propene conversion accompanying the Sₒᵥₑ values as described above will be ≦99.5 mol %, or ≦99 mol % or ≦98.5 mol %.

In addition, the aforementioned values of Sₒᵥₑ are generally accompanied by selectivities (based on the molar amount of propene converted) of acrylic acid formation S^{AA}ₚₑₙ, which are ≧80 mol %, or ≧85 mol %, or ≧>90 mol % or ≧92 mol %, frequently ≧93 mol % or a ≧94 mol %, preferably a ≧95 mol % or ≧96 mol %. Frequently, the aforementioned values of S^{AA}ₚₑₙ, will be at values of ≦99 mol %, or ≦98 mol %, or ≦97 mol %. The aforementioned values of S^{AA}ₚₑₙ may be accompanied by any of the aforementioned values of C^{pen}.

When propane is the starting material for the inventive heterogeneously catalyzed partial gas phase oxidation, the aforementioned values for Sₒᵥₑ are normally accompanied by molar propane conversions (based on single paths of the reaction gas mixture through all reaction stages) C^{pan} which are ≧20 mol %, or ≧25 mol %, or ≧30 mol %, or ≧35 mol %, or ≧40 mol %, or ≧45 mol %, or ≧50 mol %, or ≧55 mol %, or ≧60 mol %, or ≧65 mol %, or ≧70 mol %, or ≧75 mol %, or ≧80 mol %, or ≧85 mol %. Frequently, the aforementioned values of C^{pan} will be ≦95 mol %, in many cases ≦90 mol %.

In addition, the aforementioned values of Sₒᵥₑ are generally accompanied by selectivities (based on the molar amount of propane converted) of acrylic acid formation S^{AA}ₚₐₙ which are ≧50 mol %, or ≧55 mmol %, or ≧60 mol %, preferably ≧65 mol % and more preferably ≧70 mol % or ≧75 mol %. Frequently, the aforementioned values of S^{AA}ₚₐₙ will be at values of ≦95 mol %, or ≦90 mol % or ≦85 mol %.

It is appropriate from an application point of view to carry out the process according to the invention in a particularly simple manner in such a way that a conventional process, as described in the prior art, of a heterogeneously catalyzed partial oxidation of at least one C₃ hydrocarbon precursor compound (may also be a mixture of propane and propene) to acrylic acid is initially carried out (main reaction). For example, this conventional process (the main reaction) may be carried out as described in the documents WO 01/96270, DE-A 10344264, DE-A 10353954, DE-A 10313213, DE-A 19927624, DE-A 10254279, DE-A 10344265, DE-A 10246119, DE-A 10245585, DE-A 10313208, DE-A 10303526, DE-A 10302715, DE-A 10261186, DE-A 10254279, DE-A 10254278, DE-A 10316465 and the prior art cited therein.

The product gas mixture obtainable in this way and having a comparatively elevated secondary component content (for example secondary component formation ≧1.7 mol %) is subsequently, if appropriate after addition of inert gas (e.g. N₂, CO₂, steam or any mixture thereof) or of molecular oxygen or of a mixture of molecular oxygen and inert gas, in a postreaction stage at elevated temperature, conducted through a catalyst charge in such a way that the acrylic acid present in the product gas mixture remains substantially unchanged, while the secondary components are at least partly combusted to carbon oxides and water, which reduces the overall selectivity of secondary component formation (Sₒᵥₑ) over the entire process (preference is given in accordance with the invention to Sₒᵥₑ (in mol %) falling by at least 0.3, preferably by at least 0.5, more preferably by at least 0.8 and most preferably by at least 1, percent or more), without significantly impairing the selectivity of acrylic acid formation (preference is given in accordance with the invention to the selectivity of acrylic acid formation (S^{AA}ₚₑₙ or S^{AA}ₚₐₙ, each in mol %) falling by less than 2, preferably by less than 1.5, more preferably by less than 1, even more preferably by less than 0.8, better by less than 0.6, even better by less than 0.4 and even better by less than 0.2 or 0.1 (or no) percent). In favorable cases, the selectivity of acrylic acid formation even increases by up to 1, or by up to 0.5 or 0.3 percent. The aforementioned percentage changes of Sₒᵥₑ and S^{AA}ₚₑₙ or S^{AA}ₚₐₙ may be realized in accordance with the invention in any desired combination.

The overall selectivity of secondary component formation Sₒᵥₑ in the main reaction is normally at values of ≧1.7 mol % or ≧1.8 mol % or ≧1.9 mol %.

When propene is the starting material for the inventive heterogeneously catalyzed partial gas phase oxidation, the aforementioned main reaction values of Sₒᵥₑ are normally accompanied by molar main reaction propene conversions C^{pen} which are ≧95 mol %, or ≧96 mol %, preferably ≧98 mol % (based in each case on the starting amount of propene). Frequently, the propene conversion accompanying the Sₒᵥₑ values of the main reaction as described above will be ≦99.5 mol %, or ≦99 mol % or ≦98.5 mol %. In addition, the aforementioned values of Sₒᵥₑ in the main reaction are generally accompanied by selectivities (based on the molar amount of propene converted in the main reaction) of acrylic acid formation S^{AA}ₚₑₙ which are ≧80 mol %, or ≧85 mol %, or ≧90 mol %, or ≧92 mol %, frequently ≧93 mol %, or ≧94 mol %, preferably ≧95 mol % or ≧96 mol %. Frequently, the aforementioned values of S^{AA}ₚₑₙ of the main reaction will be at values of ≦99 mol %, or ≦98 mol %, or ≦97 mol %. The aforementioned values of S^{AA}ₚₑₙ of the main reaction may be accompanied by any of the aforementioned values of C^{pen} for the main reaction.

When propane is the starting material for the inventive heterogeneously catalyzed partial gas phase oxidation, the aforementioned main reaction values of Sₒᵥₑ are normally accompanied in the main reaction by molar propane conversions C^{pan} which are ≧20 mol %, or ≧25 mol %, or ≧30 mol %, or ≧35 mol %, or ≧40 mol %, or ≧45 mol %, or ≧50 mol %, or ≧55 mol %, or ≧60 mol %, or ≧65 mol %, or ≧70 mol %, or ≧75 mol %, or ≧80 mol %, or ≧85 mol %. Frequently, the aforementioned values for C^{pan} of the main reaction will be ≦95 mol %, in many cases ≦90 mol %. In addition, the aforementioned values of Sₒᵥₑ of the main reaction are generally accompanied by selectivities (based on the molar amount of propane converted in the main reaction) of acrylic acid formation S^{AA}ₚₐₙ which are ≧50 mol %, or ≧55 mol %, or ≧60 mol %, preferably ≧65 mol % and more preferably ≧70 mol % or ≧75 mol %. Frequently, the aforementioned values of S^{AA}ₚₐₙ of the main reaction will be at values of ≦95 mol %, or ≦90 mol % or ≦85 mol %.

The active composition of catalysts of such a postreaction stage (postcatalysts) may, for example, be multimetal oxide compositions of the general formula I
Mo₁₂VₐX¹_{b}X²_{c}X³_{d}X⁴ₑX⁵_{f}X⁶_{g}X⁷ₕOₙ (I)
where
X¹=W, Nb, Ta, Cr and/or Ce, preferably W,
X²=Cu, Ni, Co, Fe, Mn and/or Zn, preferably Cu and/or Fe,
X³=Sb and/or Bi, preferably Sb,
X⁴=one or more alkali metals,
X⁵=one or more alkaline earth metals,
X⁶=Si, Al, Ti and/or Zr,
X⁷=Pd, Pt, Ag, Rh and/or Ir, preferably Pd,
a=from 1 to 6,
b=from 0.2 to 4,
c=from 0.5 to 18, preferably from 0.5 to 5 and more preferably from 2 to 5,
d=from 0 to 40, preferably from >0 to 40 and more preferably from 0.5 to 10,
e=from 0 to 2,
f=from 0 to 4,
g=from 0 to 40,
h=from 0 to 1, preferably from >0 to 1 and more preferably from 0.01 to 0.05, and
n=a number which is determined by the valency and frequency of the elements in I other than oxygen.

Embodiments preferred in accordance with the invention within the active multimetal oxides I are those which are encompassed by the following definitions of the variables of the general formula I:
X¹=W, Nb and/or Cr, preferably W,
X²=Cu, Ni, Co and/or Fe, preferably Cu and/or Fe,
X³=Sb and/or Bi,
X⁴=Na and/or K,
X⁵=Ca, Sr, and/or Ba,
X⁶=Si, Al and/or Ti,
X⁷=Pd, Pt, Ag, Rh and/or Ir, preferably Pd
a=from 1.5 to 5,
b=from 0.5 to 2,
c=from 0.5 to 5, preferably from 2 to 5,
d=from 0 to 2, preferably from 0.5 to 2,
e=from 0 to 2,
f=from 0 to 4,
g=from 0 to 40,
h=from 0 to 1, preferably from >0 to 1 and more preferably from 0.01 to 0.05, and
n=a number which is determined by the valency and frequency of the elements in I other than oxygen.

However, multimetal oxides I which are very particularly preferred in accordance with the invention are those of the general formula II
Mo₁₂V_{a′}Y¹_{b′}Y²_{c′}Y³_{d′}Y⁴_{e′}Y⁵_{f′}Y⁶_{g′}O_{n,} (II)
where
Y¹=W and/or Nb, preferably W,
Y²=Cu and/or Fe,
Y³=Sb and/or Bi,
Y⁴=Ca and/or Sr,
Y⁵=Si and/or Al,
Y⁶=Pd, Pt, Ag, Rh and/or Ir, preferably Pd,
a′=from 2 to 4,
b′=from 1 to 1.5,
c′=from 2 to 5,
d′=from 0 to 2, preferably from 0.5 to 2,
e′=from 0 to 0.5,
f′=from 0 to 8,
g′=from 0 to 1, preferably from >0 to 1 and more preferably from 0.01 to 0.05, and
n′=a number which is determined by the valency and frequency of the elements in II other than oxygen.

In principle, multimetal oxide active compositions I can be prepared in a simple manner by obtaining a very intimate, preferably finely divided dry mixture having a composition corresponding to their stoichiometry from suitable sources of their elemental constituents and calcining it at temperatures of from 350 to 600° C. The calcination may be carried out either under inert gas or under an oxidative atmosphere, for example air (mixture of inert gas and oxygen), and also under a reducing atmosphere (for example mixtures of inert gas and reducing gases such as H₂, NH₃, CO, methane and/or acrolein or the reducing gases mentioned themselves). The calcination time can be from a few minutes to a few hours and typically falls with temperature. Preference is given in accordance with the invention to effecting the calcination (and also the catalyst preparation overall) as described in DE-A 10360057 or DE-A 10360058. Useful sources for the elemental constituents of the multimetal oxide active compositions I include those compounds which are already oxides and/or those compounds which can be converted to oxides by heating, at least in the presence of oxygen.

The starting compounds for preparing multimetal oxide compositions I can be intimately mixed in dry or in wet form. When they are mixed in dry form, the starting compounds are advantageously used in the form of finely divided powder and subjected to calcining after mixing and, if appropriate, compaction. However, preference is given to effecting the intimate mixing in wet form.

This is typically done by mixing the starting compounds in the form of an aqueous solution and/or suspension. Particularly intimate dry mixtures are obtained in the mixing process described when the starting materials are exclusively sources of the elemental constituents in dissolved form. The solvent used is preferably water. Subsequently, the aqueous composition obtained is dried, and the drying operation is preferably effected by spray-drying the aqueous mixture at outlet temperatures of from 100 to 150° C.

The resulting multimetal oxide compositions I may be used for the process according to the invention either in powder form or shaped to certain catalyst geometries, and the shaping may be effected before or after the final calcination. For example, unsupported catalysts can be prepared from the powder form of the active composition or its uncalcined precursor composition by compacting to the desired catalyst geometry (for example by tableting or extruding), if appropriate with the addition of assistants, for example graphite or stearic acid as lubricants and/or shaping assistants, and reinforcing agents such as microfibers of glass, asbestos, silicon carbide or potassium titanate. Examples of suitable unsupported catalyst geometries are solid cylinders or hollow cylinders having an external diameter and a length of from 2 to 10 mm. In the case of the hollow cylinder, a wall thickness of from 1 to 3 mm is advantageous. It will be appreciated that the unsupported catalyst may also have spherical geometry, in which case the spherical diameter may be from 2 to 10 mm.

It will be appreciated that the pulverulent active composition or its pulverulent precursor composition which is yet to be calcined can also be shaped by applying to preshaped inert catalyst supports. The coating of the support bodies to prepare the coated catalysts is generally performed in a suitable rotatable vessel, as disclosed, for example, by DE-A 2909671, EP-A 293859 or by EP-A 714700.

To coat the support bodies, the powder composition to be applied is appropriately moistened and is dried again after application, for example by means of hot air. The layer thickness of the powder composition applied to the support body is appropriately selected within the range from 10 to 1000 μm, preferably within the range from 50 to 500 μm and more preferably within the range from 150 to 250 μm.

Useful support materials are customary porous or nonporous aluminum oxides, silicon dioxide, thorium dioxide, zirconium dioxide, silicon carbide or silicates such as magnesium silicate or aluminum silicate. The support bodies may have a regular or irregular shape, although preference is given to regularly shaped support bodies having distinct surface roughness, for example spheres or hollow cylinders having a grit layer. It is suitable to use substantially nonporous, surface-roughened (cf. DE-A 2135620), spherical supports made of steatite (especially made of Steatite C220 from CeramTec) whose diameter is from 1 to 8 mm, preferably from 4 to 5 mm. However, suitable support bodies also include corresponding cylinders whose length is from 2 to 10 mm and whose external diameter is from 4 to 10 mm. In the case of rings as support bodies, the wall thickness is also typically from 1 to 4 mm. Annular support bodies to be used with preference have a length of from 2 to 6 mm, an external diameter of from 4 to 8 mm and a wall thickness of from 1 to 2 mm.

Suitable support bodies are also in particular rings of geometry 7 mm×3 mm×4 mm (external diameter×length×internal diameter). It will be appreciated that the fineness of the catalytically active oxide compositions to be applied to the surface of the support body is adapted to the desired coating thickness (cf. EP-A 714700, DE-A 10360057, DE-A 10360058).

In addition to the element oxides, useful sources for preparing multimetal oxides I are in particular halides, nitrates, formates, oxalates, citrates, acetates, carbonates, amine complexes, ammonium salts and/or hydroxides of the desired elemental constituents (compounds such as NH₄OH, (NH₄)₂CO₃, NH₄NO₃, NH₄CHO₂, CH₃COOH, NH₄CH₃CO₂ and/or ammonium oxalate which, on subsequent calcining at the latest, decompose to give compounds which escape in gaseous form and/or can be decomposed may additionally be incorporated into the intimate dry mixture).

However, it will be appreciated that it is also possible to preform, from partial amounts of elements, multimetal oxide compositions which then find use as element sources for preparing multimetal oxides I. Appropriate preparation processes are described, for example, in EP-A 668104, DE-A 19736105, DE-A 10046928, DE-A 19740493 and in DE-A 19528646. For example, such a multimetal oxide composition for partial amounts is FeSb₂O₆.

However, useful active compositions of catalysts of an inventive postreaction stage also include multimetal oxide compositions of the general formula III
MO₁VₐM¹_{b}M²_{c}M³_{d}Oₙ (III)
where
M¹=at least one of the elements from the group consisting of Te and Sb;
M²=at least one of the elements from the group consisting of Nb, Ti, W, Ta and Ce;
M³=at least one of the elements from the group consisting of Pb, Ni, Co, Bi, Pd, Ag, Pt, Cu, Au, Ga, Zn, Sn, In, Re, Ir, Sm, Sc, Y, Pr, Nd and Tb;
a=from 0.01 to 1,
b=from >0 to 1,
c=from >0 to 1,
d=from 0 to 0.5, preferably from >0 to 0.5, and
n=a number which is determined by the valency and frequency of the elements in III other than oxygen,
   and an x-ray diffractogram which has reflections h, i and k whose peak locations are at the reflection angles (2⊖) of 22.2±0.5° (h), 27.3±0.5° (i) and 28.2±0.5° (k), and
   the reflection h has the highest intensity within the x-ray diffractogram and a half-height width of at most 0.5°,
   the intensity Pᵢ of the reflection i and the intensity Pₖ of the reflection k satisfy the relationship 0.20≦R≦0.85 in which R is the intensity ratio defined by the formula
      R=Pᵢ/(Pᵢ+Pₖ),
   and
   the half-height width of the reflection i and of the reflection k are each ≦1°.

Preferably in accordance with the invention, 0.3≦R≦0.85, better 0.4≦R≦0.85, more preferably 0.65≦R≦0.85, still more preferably 0.67≦R≦0.75 and even more preferably R=0.69 to 0.75 or R=from 0.71 to 0.74 or R=0.72.

It is also advantageous for the process according to the invention when the multimetal oxide composition III is one whose x-ray diffractogram does not have any reflection having the peak location 2⊖=50.0±0.3° (i phase).

All data relating to an X-ray diffractogram in this document relate to an X-ray diffractogram generated using Cu-Kα radiation as the X-radiation (Siemens Theta-Theta D-5000 diffractometer, tube voltage: 40 kV, tube current: 40 mA, aperture V20 (variable), collimator V20 (variable), secondary monochromator aperture (0.1 mm), detector aperture (0.6 mm), measuring interval (2⊖): 0.02°, measuring time per step: 2.4 s, detector: scintillation counting tube; definition of the intensity of a reflection in the X-ray diffractogram relates in this document to the definition laid down in DE-A 19835247, DE-A 10122027, and also in DE-A 10051419 and DE-A 10046672; the same applies to the definition of the half-height width).

In addition to the reflections h, i and k, the X-ray diffractogram of multimetal oxide compositions (III) to be used in accordance with the invention generally also contains further reflections whose peak locations are at the following reflection angles (2⊖):
9.0±0.4° (l),
6.7±0.4° (o) and
7.9±0.4° (p).

It is also favorable when the X-ray diffractogram additionally contains a reflection whose peak location is at the reflection angle (2⊖)=45.2±0.4° (q).

Frequently, the X-ray diffractogram of multimetal oxide compositions (III) also contains the reflections 29.2±0.4° (m) and 35.4±0.4° (n) (peak locations).

When the intensity of 100 is assigned to the reflection h, it is favorable in accordance with the invention when the reflections i, l, m, n, o, p, q in the same intensity scale have the following intensities:
i: from 5 to 95, frequently from 5 to 80, in some cases from 10 to 60;
l: from 1 to 30;
m: from 1 to 40;
o: from 1 to 30;
p: from 1 to 30 and
q: from 5 to 60.

When the X-ray diffractogram of the multimetal oxide compositions (III) which are advantageous in accordance with the invention contains the aforementioned additional reflections, the half-height width thereof is generally ≦1°.

The specific surface area of multimetal oxide compositions (III) to be used in accordance with the invention is in many cases from 1 to 40 m²/g, often from 11 or 12 to 40 m²/g and frequently from 15 or 20 to 40 or 30 m²/g (determined by the BET method, nitrogen).

According to the invention, the stoichiometric coefficient a of multimetal oxide compositions (III) suitable in accordance with the invention, irrespective of the preferred ranges for the other stoichiometric coefficients of the multimetal oxide compositions (III), is preferably from 0.05 to 0.6, more preferably from 0.1 to 0.6 or 0.5.

Irrespective of the preferred ranges for the other stoichiometric coefficients of the multimetal oxide compositions (III), the stoichiometric coefficient b is preferably from 0.01 to 1, and more preferably from 0.01 or 0.1 to 0.5 or 0.4.

The stoichiometric coefficient c of the multimetal oxide compositions (III) to be used in accordance with the invention, irrespective of the preferred ranges for the other stoichiometric coefficients of the multimetal oxide compositions (III), is advantageously from 0.01 to 1 and more preferably from 0.01 or 0.1 to 0.5 or 0.4. A very particularly preferred range for the stoichiometric coefficient c which can be combined, irrespective of the preferred ranges for the other stoichiometric coefficients of the multimetal oxide compositions (III) to be used in accordance with the invention, with all other preferred ranges in this document is the range from 0.05 to 0.2.

According to the invention, the stoichiometric coefficient d of multimetal oxide compositions (III) suitable in accordance with the invention, irrespective of the preferred ranges for the other stoichiometric coefficients of the multimetal oxide compositions (III), is preferably from 0.00005 or 0.0005 to 0.5, more preferably from 0.001 to 0.5, frequently from 0.002 to 0.3 and often from 0.005 or 0.01 to 0.1.

Particularly favorable for the process according to the invention are multimetal oxide compositions (III) whose stoichiometric coefficients a, b, c and d are simultaneously within the following framework:
a=from 0.05 to 0.6;
b=from 0.01 to 1 (or from 0.01 to 0.5);
c=from 0.01 to 1 (or from 0.01 to 0.5); and
d=from 0.0005 to 0.5 (or from. 0.001 to 0.3).

Very particularly favorable are inventive multimetal oxide compositions (III) whose stoichiometric coefficients a, b, c and d are simultaneously within the following framework:
a=from 0.1 to 0.6;
b=from 0.1 to 0.5;
c=from 0.1 to 0.5; and
d=from 0.001 to 0.5, or from 0.002 to 0.3, or from 0.005 to 0.1.

M¹ is preferably Te.

All of the aforementioned applies in particular when at least 50 mol % of the total amount of M² is Nb and most preferably when at least 75 mol % of the total amount of M², or 100 mol % of the total amount of M², is Nb.

It also applies in particular, irrespective of the definition of M², when M³ is at least one element from the group consisting of Ni, Co, Bi, Pd, Ag, Au, Pb and Ga, or at least one element from the group consisting of Ni, Co, Pd and Bi.

All of the aforementioned also applies in particular when at least 50 mol % of the total amount of M², or at least 75 mol %, or 100 mol %, is Nb, and M³ is at least one element from the group consisting of Ni, Co, Bi, Pd, Ag, Au, Pb and Ga.

All of the aforementioned also applies in particular when at least 50 mol %, or at least 75 mol %, or 100 mol %, of the total amount of M² is Nb, and M³ is at least one element from the group consisting of Ni, Co, Pd and Bi.

Very particular preference is given to all statements regarding the stoichiometric coefficients applying when M¹=Te, M²=Nb and M³=at least one element from the group consisting of Ni, Co and Pd.

Preparation processes for multimetal oxide active compositions III can be found in the prior art. These include in particular DE-A 10122027, DE-A 10119933, DE-A 10033121, EP-A 1192987, DE-A 10029338, JP-A 2000-143244, EP-A 962253, EP-A 895809, DE-A 19835247, WO 00/29105, WO 00/29106, EP-A 529853 and EP-A 608838 (the drying method employed in all working examples of the last two documents is spraydrying; for example with an inlet temperature of from 300 to 350° C. and an outlet temperature of from 100 to 150° C.; countercurrent or cocurrent).

The principle of a selective process for preparing multimetal oxide compositions III having i phase structure (no reflection in the x-ray diffractogram having the peak location 2⊖=50.0±0.3°) is disclosed, for example, by WO 02/06199 and the literature references cited in this document. According to these, a multimetal oxide composition (precursor multimetal oxide) is initially obtained in a manner known per se and has the stoichiometry III but a generally intimately intertwined mixed crystal system composed of i phase and other phases (for example k phase). From this mixture, the proportion of i phase can then be isolated by washing out the other phases, for example the k phase, with suitable liquids. Useful such liquids are, for example, aqueous solutions of organic acids (e.g. oxalic acid, formic acid, acetic acid, citric acid and tartaric acid), inorganic acids (e.g. nitric acid), alcohols and aqueous hydrogen peroxide solutions. In addition, JP-A 7-232071 also discloses a process for preparing i phase multimetal oxide compositions.

Multimetal oxides III which are mixed crystal systems composed of i and other (n), for example k phase, are generally obtained by the preparation processes described in the following prior art (cf., for example, DE-A 19835247, EP-A 529853, EP-A 603836, EP-A 608838, EP-A 895809, DE-A 19835247, EP-A 962253, EP-A 1080784, EP-A 1090684, EP-A 1123738, EP-A 1192987, EP-A 1192986, EP-A 1192982, EP-A 1192983 and EP-A 1192988). In these processes, a very intimate, preferably finely divided, dry mixture is generated from suitable sources of the elemental constituents of the multimetal oxide composition and thermally treated at temperatures of from 350 to 700° C. or from 400 to 650° C. or from 400 to 600° C. The thermal treatment may in principle be effected either under an oxidizing, a reducing or under an inert atmosphere. A useful oxidizing atmosphere is, for example, air, air enriched with molecular oxygen or air depleted in oxygen. However, preference is given to carrying out the thermal treatment under an inert atmosphere, i.e., for example, under molecular nitrogen and/or noble gas. Typically, the thermal treatment is effected at atmospheric pressure (1 atm). It will be appreciated that the thermal treatment may also be effected under reduced pressure or under elevated pressure.

When the thermal treatment is effected under a gaseous atmosphere, it may either be stationary or flow. It preferably flows. Overall, the thermal treatment may take up to 24 h or more.

Preference is given to effecting the thermal treatment initially under an oxidizing (oxygen-containing) atmosphere (for example under air) at a temperature of from 150 to 400° C. or from 250 to 350° C. (=predecomposition step). Afterward, the thermal treatment is appropriately continued under inert gas at temperatures of from 350 to 700° C. or from 400 to 650° C. or from 450 to 600° C. It will be appreciated that the thermal treatment may also be effected in such a way that the catalyst precursor composition, before its thermal treatment, is initially (optionally after pulverization) tableted (optionally with the addition of from 0.5 to 4 or 2% by weight of finely divided graphite), then thermally treated and subsequently spalled again.

The intimate mixing of the starting compounds may be effected in dry or in wet form.

When it is effected in dry form, the starting compounds are appropriately used as finely divided powder and, after the mixing and any compaction, subjected to calcination (thermal treatment).

However, preference is given to effecting the intimate mixing in wet form. Typically, the starting compounds are mixed together in the form of an aqueous solution (if appropriate with the use of complexing agents; cf., for example, DE-A 10145958) and/or suspension. Subsequently, the aqueous composition is dried and calcined after the drying. Appropriately, the aqueous composition is an aqueous solution or an aqueous suspension. Preference is given to effecting the drying process directly after the preparation of the aqueous mixture (especially in the case of an aqueous solution; cf., for example, JP-A 7-315842) and by spray drying (the exit temperatures are generally from 100 to 150° C.; the spray drying may be carried out in cocurrent or in countercurrent), which results in a particularly intimate dry mixture, in particular when the aqueous composition to be spray-dried is an aqueous solution or suspension. However, it may also be dried by concentrating by evaporation under reduced pressure, by freeze-drying or by conventional concentration by evaporation.

Useful sources for the elemental constituents when carrying out the above-described preparation method of i/k phase mixed crystal multimetal oxide compositions are, for example, all of those which are capable of forming oxides and/or hydroxides on heating (if appropriate under air). It will be appreciated that such starting compounds may also partly or exclusively be oxides and/or hydroxides of the elemental constituents. In other words, useful starting compounds are especially all of those mentioned in the documents of the acknowledged prior art, also including DE-A 10254279.

The i/k phase mixed crystal multimetal oxide compositions, for example, obtainable as described (pure i phase multimetal oxides are obtained coincidentally if at all by the procedure described) may then be converted to i phase multimetal oxides (III) suitable in accordance with the invention by suitable washing (for example in accordance with DE-A 10254279).

An increased proportion of i phase (and in favorable cases substantially pure i phase) is established in the preparation of multimetal oxides which can be converted by washing described to multimetal oxides (III) suitable in accordance with the invention when they are prepared by a hydrothermal route, as described, for example, in DE-A 10029338 and JP-A 2000-143244.

However, multimetal oxide compositions (III) to be used in accordance with the invention can also be prepared by initially generating a multimetal oxide composition III′ which differs from a multimetal oxide composition (III) only in that d=0.

Such a preferably finely divided multimetal oxide composition III′ may then be saturated with solutions (for example aqueous) of elements M³ (for example by spraying), subsequently dried (preferably at temperatures ≦100° C.) and then, as already described for the precursor multimetal oxides, calcined (preferably in an inert gas stream; preference is given here to dispensing with predecomposition under air). The use of aqueous nitrate and/or halide solutions of elements M³ and/or the use of aqueous solutions in which the elements M³ are complexed with organic compounds (for example acetates or acetylacetonates) is particularly advantageous for this preparative variant.

The multimetal oxides (III) obtainable as described may be used for the inventive postreaction stage as such [for example as a powder or after tableting the powder (frequently with the addition of from 0.5 to 2% by weight of finely divided graphite) and subsequent spalling to give spall] or else shaped to shaped bodies.

The shaping to shaped bodies may be effected, for example, by applying to a support body, as described in DE-A 10118814, or PCT/EP/02/04073, or DE-A 10051419.

According to the invention, useful materials for the support bodies are in particular aluminum oxide, silicon dioxide, silicates such as clay, kaolin, steatite (preferably having a low water-soluble alkali content), pumice, aluminum silicate and magnesium silicate, silicon carbide, zirconium dioxide and thorium dioxide. Preference is given to Steatite C220 from CeramTec, and support bodies having a core substantially free of pores and only a porous coating are particularly advantageous.

The surface of the support body may be either smooth or rough. Advantageously, the surface of the support body is rough, since increased surface roughness generally results in increased adhesion of the applied active composition coating (cf., for example, DE-A 2135620).

Frequently, the surface roughness R_{z} of the support body is in the range from 5 to 200 μm, often in the range from 20 to 100 μm (determined according to DIN 4768 sheet 1 using a “Hommel tester for DIN-ISO surface parameters” from Hommelwerke, Germany).

In addition, the support material may be porous or nonporous. Appropriately, the support material is nonporous (total volume of the pores based on the volume of the support body ≦1% by volume). Preference is given to support material having a nonporous core and porous coating (cf. DE-A 2135620).

The thickness of active oxide composition III coatings on coated catalysts to be used in accordance with the invention is typically from 10 to 1000 μm. However, it may also be from 50 to 700 μm, from 100 to 600 μm or from 150 to 400 μm. Possible coating thicknesses are also from 10 to 500 μm, from 100 to 500 μm or from 150 to 300 μm.

In principle, any geometries of the support bodies are useful for the process according to the invention. Their longest dimension is generally from 1 to 10 mm. However, preference is given to using spheres or cylinders, in particular hollow cylinders, as support bodies. Favorable diameters for support spheres are from 1.5 to 4 mm. When the support bodies used are cylinders, their length is preferably from 2 to 10 mm and their external diameter is preferably from 4 to 10 mm. In the case of rings, the wall thickness is additionally typically from 1 to 4 mm. Annular support bodies which are suitable in accordance with the invention may also have a length of from 3 to 6 mm, an external diameter of from 4 to 8 mm and a wall thickness of from 1 to 2 mm. However, a support ring geometry of 7 mm×3 mm×4 mm or of 5 mm×3 mm×2 mm (external diameter×length×internal diameter) is also possible.

Such coated catalysts to be used in accordance with the invention can be prepared in the simplest manner, for example, in such a way that oxide compositions of the general formula (III) to be used in accordance with the invention are preformed, they are converted to a finely divided form and finally applied to the surface of the support body with the aid of a liquid binder. To this end, the surface of the support body is, in the simplest manner, moistened with the liquid binder and a layer of the active composition is attached to the moistened surface by contacting with the finely divided active oxide composition of the general formula (III). Finally, the coated support body is dried. It will be appreciated that the procedure may be repeated periodically to achieve an increased layer thickness. In this case, the coated basic body becomes the new “support body”, etc.

The fineness of the catalytically active oxide composition of the general formula (III) to be applied to the surface of the support body is of course adapted to the desired coating thickness. Suitable for the coating thickness range of from 100 to 500 μm are, for example, those active composition powders of which at least 50% of the total number of powder particles pass through a sieve of mesh width from 1 to 20 μm and whose numerical fraction of particles having a longest dimension of above 50 μm is less than 10%. In general, the distribution of the longest dimensions of the powder particles, as a result of the preparation, corresponds to a Gaussian distribution. Frequently, the particle size distribution is as follows:

| | D (μm) | | | | | | | | | | | | | | |
|---|---|---|---|---|---|---|---|---|---|---|---|---|---|---|---|
| | 1 | 1.5 | 2 | 3 | 4 | 6 | 8 | 12 | 16 | 24 | 32 | 48 | 64 | 96 | 128 |
| x | 80.5 | 76.3 | 67.1 | 53.4 | 41.6 | 31.7 | 23 | 13.1 | 10.8 | 7.7 | 4 | 2.1 | 2 | 0 | 0 |
| y | 19.5 | 23.7 | 32.9 | 46.6 | 58.4 | 68.3 | 77 | 86.9 | 89.2 | 92.3 | 96 | 97.9 | 98 | 100 | 100 |

In this table:
D=diameter of the particle,
x=the percentage of the particles whose diameter is 2 D; and
y=the percentage of the particles whose diameter is <D.

For a performance of the coating process described on the industrial scale, it is recommended, for example, to employ the process principle disclosed in DE-A 2909671, and also in DE-A 10051419. In other words, the support bodies to be coated are initially charged in a preferably inclined (the inclination angle is generally ≧0° and ≦90°, usually ≧300 and ≦90°; the inclination angle is the angle of the central axis of the rotating vessel relative to the horizontal) rotating vessel (for example rotary pan or coating drum). The rotating vessel conducts the, for example, spherical or cylindrical support bodies under two metering devices arranged successively in a certain separation. The first of the two metering devices appropriately corresponds to a nozzle (for example an atomizer nozzle operated with compressed air), which sprays the support bodies rolling in the rotating rotary pan with the liquid binder and moistens them in a controlled manner. The second metering device is disposed outside the atomization cone of the sprayed liquid binder and serves to feed the finely divided oxidic active composition (for example via an agitated channel or a powder screw). The support spheres which have been moistened in a controlled manner take up the active composition powder supplied, which is compressed by the rolling motion on the outer surface of the, for example, cylindrical or spherical support body to give a continuous coating.

If required, the support body basically coated in this way, in the course of the subsequent rotation, again passes through the spray nozzles, and is moistened in a controlled manner, in order, in the course of the further motion, to be able to take up a further layer of finely divided oxidic active composition, etc. (intermediate drying is generally not necessary). Finely divided oxidic active composition and liquid binder are generally supplied continuously and simultaneously.

The liquid binder may be removed on completion of coating, for example by the action of hot gases such as N₂ or air. Remarkably, the coating process described brings about fully satisfactory adhesion of the successive layers both to each other and to the base layer on the surface of the support body.

It is essential for the above-described coating method that the moistening of the surface of the support body to be coated is undertaken in a controlled manner. In short, this means that the support surface is appropriately moistened in such a way that, although it has adsorbed liquid binder, no liquid phase as such visibly appears on the support surface. When the support body surface is too moist, the finely divided catalytically active oxide composition agglomerates to separate agglomerates, instead of attaching to the surface. Detailed information on this subject can be found in DE-A 2909671 and in DE-A 10051419.

The aforementioned final removal of the liquid binder used can be undertaken in a controlled manner, for example by evaporation and/or sublimation. In the simplest case, this may be effected by the action of hot gases at appropriate temperature (frequently from 50 to 300° C., frequently 150° C.). However, the action of hot gases may also be used only to bring about predrying. The final drying may then be effected, for example, in a drying oven of any type (for example belt dryer) or in the reactor. The action temperature should not be above the calcination temperature employed to prepare the oxidic active composition. It will be appreciated that the drying may also be carried out exclusively in a drying oven.

The binder used for the coating operation, irrespective of the type and the geometry of the support body, may be: water, monohydric alcohols such as ethanol, methanol, propanol and butanol, polyhydric alcohols such as ethylene glycol, 1,4-butanediol, 1,6-hexanediol or glycerol, mono- or polybasic organic carboxylic acids such as propionic acid, oxalic acid, malonic acid, glutaric acid or maleic acid, amino alcohols such as ethanolamine or diethanolamine, or else mono- or polyhydric organic amides such as formamide. Favorable binders are also solutions consisting of from 20 to 90% by weight of water and from 10 to 80% by weight of an organic compound dissolved in water, whose boiling point or sublimation temperature at atmospheric pressure (1 atm) is >100° C., preferably >150° C. Advantageously, the organic compound is selected from the above listing of possible organic binders. The organic fraction of the aforementioned aqueous binder solutions is preferably from 10 to 50% by weight and more preferably from 20 to 30% by weight. Useful organic components are also mono-saccharides and oligosaccharides such as glucose, fructose, sucrose or lactose, and also polyethylene oxides and polyacrylates.

It is significant that coated catalysts suitable in accordance with the invention can be prepared not only by applying the finished, finely ground active oxide compositions of the general formula (III) to the moistened support body surface.

Rather, instead of the active oxide composition, a finely divided precursor composition thereof may also be applied to the moistened support surface (employing the same coating process and binder) and the calcination carried out after drying the coated support body (support bodies may also be impregnated with a precursor solution, subsequently dried and then calcined). Finally, the phases other than the i phase may be washed out if required.

Such a finely divided precursor composition may be, for example, that composition which is obtainable by initially generating a very intimate, preferably finely divided dry mixture from the sources of the elemental constituents of the desired active oxide composition of the general formula (III) (for example by spray drying an aqueous suspension or solution of the sources), and thermally treating this finely divided dry mixture (if appropriate after tableting with the addition of from 0.5 to 2% by weight of finely divided graphite) at a temperature of from 150 to 350° C., preferably from 250 to 350° C., under an oxidizing (oxygen-containing) atmosphere (for example under air) (for a few hours) and finally, if required, subjecting it to grinding.

After the coating of the support bodies with the precursor composition, calcination is then effected, preferably under an inert gas atmosphere (all other atmospheres are also possible), at temperatures of from 360 to 700° C. or from 400 to 650° C. or from 400 to 600° C.

It will be appreciated that multimetal oxide compositions (III) which can be used in accordance with the invention may also be shaped by extrusion and/or tableting, either of finely divided multimetal oxide composition (III) or of finely divided precursor composition of a multimetal oxide composition (III) (if necessary, the phases other than the i phase can finally be washed out).

Useful geometries are spheres, solid cylinders and hollow cylinders (rings). The longest dimension of the aforementioned geometries is generally from 1 to 10 mm. In the case of cylinders, their length is preferably from 2 to 10 mm and their external diameter is preferably from 4 to 10 mm. In the case of rings, the wall thickness is additionally typically from 1 to 4 mm. Annular unsupported catalysts suitable according to the invention may also have a length of from 3 to 6 mm, an external diameter of from 4 to 8 mm and a wall thickness of from 1 to 2 mm. However, an unsupported catalyst ring geometry of 7 mm×3 mm×4 mm or of 5 mm×3 mm×2 mm (external diameter×length×internal diameter) is also possible.

The geometry of the multimetal oxide active compositions (III) for the process according to the invention may of course also be all of those of DE-A 10101695 (what has been stated on the shaping of the multimetal oxide III catalysts can thus also be applied to the shaping of the multimetal oxide I catalysts).

In this document, the definition of the intensity of a reflection in the X-ray diffractogram relates, as already stated, to the definition laid down in DE-A 19835247, and also in DE-A 10051419 and DE-A 10046672.

In other words, if A¹ denotes the peak location of a reflection 1 and B¹, in the line of the x-ray diffractogram viewed along the intensity axis at right angles to the 2⊖ axis, denotes the next pronounced minimum (minima having reflection shoulders are not taken into account) to the left of the peak location A¹ and B² is correspondingly the next pronounced minimum to the right of the peak location A¹ and C¹ denotes the point at which a straight line drawn from the peak location A¹ at right angles to the 2⊖ axis cuts a straight line joining the points B¹ and B², the intensity of the reflection 1 is the length of the straight line section A¹C¹ which then extends from the peak location A¹ to the point C¹. The expression minimum in this context means a point at which the slope of a tangent to the curve in a base region of the reflection 1 changes from a negative value to a positive value, or a point at which the slope tends to zero, using the coordinates of the 2⊖ axis and of the intensity axis for the determination of the slope.

In this document, the half-height width is correspondingly the length of the straight line section between the two intersection points H¹ and H² when a line is drawn parallel to the 2⊖ axis in the middle of the straight line section A¹C¹, H¹, H² meaning in each case the first point at which these parallel lines cut the line as defined above of the x-ray diffractogram to the left and right of A¹.

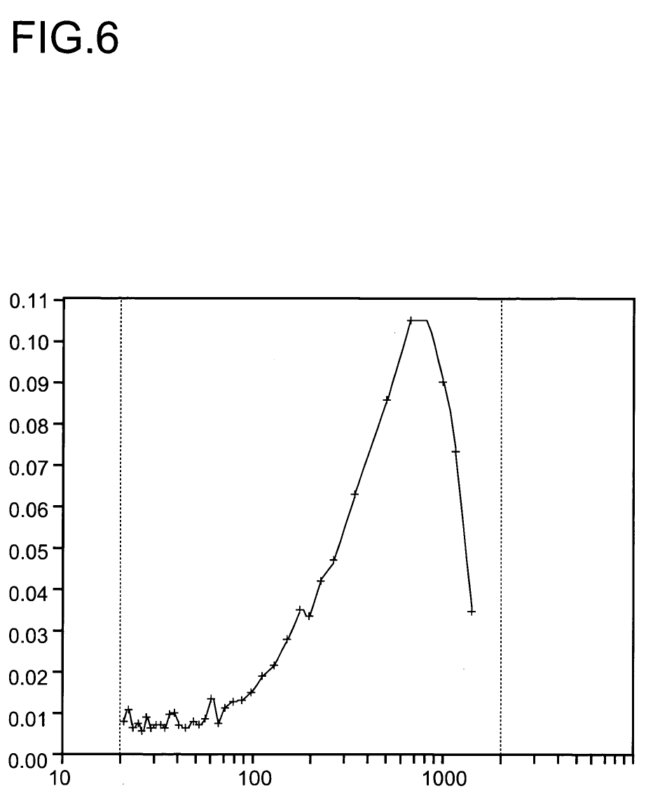

An exemplary execution of the determination of half-height width and intensity is also shown by FIG. 6 in DE-A 10046672.

It will be appreciated that the multimetal oxide compositions (III) to be used in accordance with the invention may also be used for the inventive postreaction stage in the form of catalytic active compositions diluted with finely divided, for example colloidal, materials such as silicon dioxide, titanium dioxide, aluminum oxide, zirconium oxide, niobium oxide.

The dilution composition ratio may be up to 9 (diluent):1 (active composition). In other words, possible diluent composition ratios are, for example, 6 (diluent):1 (active composition) and 3 (diluent):1 (active composition). The diluent can be incorporated before and/or after the calcination, generally even before the drying.

When the incorporation is effected before the drying or before the calcination, the diluent has to be selected in such a way that it is substantially preserved in the fluid medium or in the calcination. This is generally the case, for example, for oxides calcined at appropriately high temperatures.

Just as the conventional processes, described in the prior art, for heterogeneously catalyzed partial oxidation of at least one C₃ hydrocarbon precursor compound to acrylic acid (the main reaction) may be carried out in a fixed catalyst bed, moving bed or fluidized bed, the inventive postreaction stage may also be carried out in a fixed catalyst bed, moving bed or fluidized bed.

In the case of a partial oxidation of propene to acrylic acid, the conventional heterogeneously catalyzed partial oxidation (the main reaction) is carried out, both in the one-stage and in the two-stage embodiment, preferably in tube bundle reactors, as described, for example, in the documents DE-A 10246119 and DE-A 10245585.

When the catalyst charge is disposed in the catalyst tubes through which the reaction mixture is conducted, a heat exchange medium (generally a salt bath) is conducted around the catalyst tubes. It will be appreciated that, along the catalyst tubes of a tube bundle reactor, it is also possible to conduct a plurality of mutually independent heat exchange media around the accompanying catalyst tube sections in a spatially successive manner. In this case, reference is made to a multitemperature zone reactor.

The inventive postreaction stage may then be effected in an integrated manner either in a separate postreactor spatially downstream of the conventional partial oxidation or in a reactor of the main reaction. Suitable for the postreactor type are in principle all of those which are also suitable for the main reaction.

When the conventional heterogeneously catalyzed partial oxidation (the main reaction) of propene to acrylic acid is effected, for example, in two tube bundle reactors connected in series (of which each may have one or more temperature zones), the first tube bundle reactor substantially oxidizing propene to acrolein and the second tube bundle reactor the acrolein formed in the first tube bundle reactor to acrylic acid, it is appropriate from an application point of view to insert a postcatalyst bed upstream of the outlet of the reaction gas mixture from the second tube bundle reactor in the catalyst tubes thereof (alternatively, the end of the catalyst tube may also be coated with postcatalyst on its interior; the coating may be effected, for example, as described in DE-A 19839782). When the product gas mixture of the main reaction flows through this postcatalyst bed, the secondary component content of the product gas mixture of the main reaction is reduced in accordance with the invention. The postcatalyst bed preferably occupies a tube section in the catalyst tubes which has a separate independent temperature zone. Based on the total length of the catalyst bed for main reaction and postreaction, the postreaction bed length should be from 10 to 30%.

When the product gas mixture of the main reaction is to be supplemented with molecular oxygen or a mixture of molecular oxygen and inert gas before the postreaction is carried out, the alternative variant in which the postcatalysts are disposed in a spatially separate downstream reactor is advantageous.

This may likewise be a tube bundle reactor, but also a fluidized bed or moving bed reactor.

It is remarkable that a simple shaft furnace reactor as a fixed bed reactor, through which the hot product gas mixture of the main reaction flows axially and/or radially, if appropriate after preceding intermediate cooling, is sufficient to realize a postreaction stage, especially in adiabatic operation.

In the simplest case, this reactor is a single reaction tube whose internal diameter is from 0.1 to 10 m, possibly also from 0.5 to 5 m, and in which the fixed catalyst bed on a support device (for example a grid) is mounted. The catalyst-charged reaction tube which is thermally insulated in adiabatic operation is flowed through axially by the hot reaction gas mixture comprising the secondary components and excess molecular oxygen. The catalyst geometry may, as already mentioned, be either spherical or cylindrical or annular. However, in a manner suitable in accordance with the invention, the catalyst can also be used in spall form in the aforementioned case. To realize a radial flow of the reaction gas mixture comprising the secondary components, the postreactor may consist, for example, of two concentric cylindrical grids disposed in a jacket and the postcatalyst bed may be disposed in the annular gap between them. In the adiabatic case, the jacket would in turn be thermally insulated.

Since the postcatalysts recommended are generally also suitable for a partial oxidation of acrolein to acrylic acid (in this regard, they frequently even have an activity elevated to an exceptional extent), the acrolein content in the product gas mixture of the main reaction is also reduced simultaneously in the course of the postreaction stage to be employed as described. The content of acetic acid is usually also reduced.

Advantageously from an application point of view, the reaction temperature in the postreaction stage selected is from 200 to 300° C., frequently from 220 to 290° C. and particularly advantageously from 230 to 250° C. The reaction pressure in the postreaction stage is advantageously from 0.5 to 5 atm, usually from 1 to 3 atm. The superficial velocity on the postreaction catalyst charge with reaction gas mixture is frequently from 1500 to 2500 h⁻¹ or to 4000 h⁻¹. Normally, there is substantially no oxidation of hydrocarbons in the postreaction stage. The oxygen content of the reaction gas mixture conducted into the postreaction stage should therefore be such that it is 5 times that which is required to fully combust the secondary components including the additionally present constituents, acrolein, CO and acetic acid, present in the product gas mixture. When molecular oxygen is added to the product gas mixture of the main reaction before the postreaction, this is preferably effected in the form of air.

What has been detailed for the heterogeneously catalyzed partial oxidation of propene to acrylic acid can correspondingly also be applied to the partial oxidation of propane to acrylic acid.

It is significant according to the invention that, in the case of a multistage performance of the heterogeneously catalyzed partial oxidation of at least one C₃ hydrocarbon precursor compound to acrylic acid, a secondary component oxidation as described above (secondary component reduction) may in principle be carried out downstream of each of the reaction stages in order to lower the overall selectivity of secondary component formation over all reaction stages (overall reaction) to values of ≦1.5 mol %.

In the case of a two-stage propene partial oxidation, a large part of the secondary components formed over both reaction stages are formed in the first, the “propene-to-acrolein oxidation stage”. In other words, a secondary component oxidation inserted before the downstream “acrolein-to-acrylic acid” oxidation stage has the effect that the object of the present invention is achieved. It could also be realized, for example, in the intermediate cooler normally connected between the two oxidation stages by charging it with the postcatalyst required. Alternatively, internal surfaces of the intermediate cooler along which the reaction gas mixture to be cooled is conducted may be coated with postcatalyst. For example, such internal surfaces may be the surfaces of the cooling tubes in the case of a tube bundle heat exchanger. Frequently, for example, metal spirals are introduced into the inlet of such cooling tubes in order to improve the heat transfer. It will be appreciated that, in accordance with the invention, the surface of such spirals may appropriately likewise be coated with postcatalyst. Such coatings are capable simultaneously of counteracting carbonization of the intermediate cooler. The provision of such coatings is described, for example, in DE-A 19839782.

Aside from this, it has been found that the purer the C₃ hydrocarbon precursor compound is, the lower the overall selectivity of secondary component formation in processes for preparing acrylic acid by heterogeneously catalyzed partial oxidation of at least one C₃ hydrocarbon precursor compound. In other words, preference is given to using highly pure propene and highly pure propane for the process according to the invention. Recommended as such propane is, for example, the propane recommended in DE-A 10246119 and DE-A 10245585. In this context, propene to be used which is recommended is in particular polymer-grade propene of the following composition:
≧99.6% by weight of propene,
≦0.4% by weight of propane,
≦300 ppm by weight of ethane and/or methane,
≦5 ppm by weight of C₄ hydrocarbons,
≦1 ppm by weight of acetylene,
≦7 ppm by weight of ethylene,
≦5 ppm by weight of water,
≦2 ppm by weight of O₂,
≧2 ppm by weight of sulfur-containing compounds (calculated as sulfur),
≦1 ppm by weight of chlorine-containing compounds (calculated as chlorine),
≦5 ppm by weight of CO,
≦5 ppm by weight of CO₂,
≦10 ppm by weight of cyclopropane,
≦5 ppm by weight of propadiene and/or propyne,
≦10 ppm by weight of C_{≧5} hydrocarbon and
≦10 ppm by weight of compounds containing carbonyl groups (calculated as Ni(CO)₄).

The concept of the postreaction stage which has been introduced is all the more important when propene and/or propane comprising impurities to a significant extent is used for a heterogeneously catalyzed partial oxidation of at least one C₃ hydrocarbon precursor compound. Appropriately in accordance with the invention, an inventive postreaction stage may also be connected directly downstream of that stage (for example downstream of the catalytic dehydrogenation stage of WO 01/96270) in which propane is converted to propene. It is found that favorable postreaction stage catalysts for such a post oxidation stage are, for example, multielement oxides based on V-P-O oxides.

An alternative means of achieving the object of the present invention is to carry out the process in at least two stages and, after the reaction stage in which the propene is formed from propane, and/or after the reaction stage in which the acrolein is formed from the propene, to in each case remove the intermediate formed from the secondary components formed and to use it in thus purified form in the subsequent reaction stage. The removal may be effected, for example, as described in the documents WO 01/96270 and DE-A 10213998. Moreover, it is favorable for the process according to the invention to use catalysts in the individual reaction stages in which the desired target compound is formed particularly selectively. Such catalysts are disclosed, for example, in WO 01/96270.

It will be appreciated that the object of the present invention may be achieved by employing all measures mentioned as helpful in this document in combination or in part-combination.

The advantage of the procedure according to the invention is based on the fact that the removal of the acrylic acid from the product gas mixture can be substantially simplified. It is in principle still possible to employ the removal processes known per se and described, for example, in the documents DE-A 10235847, EP-A 792867, WO 98/01415, EP-A 1015411, EP-A 1015410, WO 99/50219, WO 00/53560, DE-A 19924532, WO 02/09839, DE-A 10235847, WO 03/041833, DE-A 10223058, DE-A 10243625, DE-A 10336386, EP-A 854 129, U.S. Pat. No. 4,317,926, DE-A 19837520, DE-A 19 606 877, DE-A 19 501 325, DE-A 10247240, DE-A 19 924 532, EP-A 982 289, DE-A 19 740 253, DE-A 19 740 252, EP-A 695 736, EP-A 982 287 and EP-A 1 041 062 (residual gas remaining may be recycled into the main reaction as cycle gas in a manner known per se). However, in apparatus terms, they may have a substantially smaller construction (especially the separating columns) and/or simpler and thus less expensive construction. For example, after the process according to the invention has been carried out, an absorptive removal of acrylic acid (absorbent may, for example, be water, a high-boiling organic solvent or else acrylic acid itself or another liquid) may, owing to the reduced polymerization tendency as a consequence of the reduced secondary component content, be carried out, instead of in a tray column, in a simplified manner in cocurrent or countercurrent version, in a randomly packed column which may be charged, for example, with Raschig rings. Alternatively, it is possible to work with the same design and a higher throughput (for example less reflux into the rectification columns). In the most favorable case in accordance with the invention, acrylic acid for preparing superabsorbents can be prepared from the product gas mixture of the gas phase oxidation directly by freezing-out on a cooling finger or condensing-out, if appropriate by means of fractional condensation. The process according to the invention may also be employed especially when the heterogeneously catalyzed main partial oxidation is a two-stage (the first stage leads substantially from propene to acrolein and the second stage leads substantially from acrolein to acrylic acid) partial oxidation of propene to acrylic acid over, for example, a fixed catalyst bed in, for example, two successive reactors, for example, tube bundle reactors, in which case both the hourly space velocity on the (fixed) catalyst bed of the first reaction stage with propene and the hourly space velocity on the (fixed) catalyst bed of the second reaction stage with acrolein are in the range of ≧120 l (STP)/l/h, or ≧130 l (STP)/l/h, or ≧140 l (STP)/l/h, or ≧150 l (STP)/l/h and ≦300 l (STP)/l/h, i.e. is carried out as what is known as a high-load process. It will be appreciated that the process according to the invention can also be employed in the aforementioned configuration when both hourly space velocities are ≦100 l (STP)/l/h.

Preference is given to carrying out each of the two (high-load) reaction stages in a reactor having more than one temperature zone (preferably two temperature zones) (cf., for example, DE-A 10313213 and DE-A 10313212).

The propene used may also be chemical-grade propene of the following specification:
≧94% by weight of propene,
≦6% by weight of propane,
≦0.2% by weight of methane and/or ethane,
≦5 ppm by weight of ethylene,
≦1 ppm by weight of acetylene,
≦20 ppm by weight of propadiene and/or propyne,
≦100 ppm by weight of cyclopropane,
≦50 ppm by weight of butene,
≦50 ppm by weight of butadiene,
≦200 ppm by weight of C₄ hydrocarbons,
≦10 ppm by weight of C_{≧5} hydrocarbons,
≦2 ppm by weight of sulfur-containing compounds (calculated as sulfur),
≦0.1 ppm by weight of sulfides (calculated as H₂S),
≦1 ppm by weight of chlorine-containing compounds (calculated as chlorine),
≦1 ppm by weight of chlorides (calculated as Cl^{⊖}) and
≦30 ppm by weight of water.

Otherwise, the high-load main partial oxidation may be carried out as described in the documents DE-A 10313213 and DE-A 10313208.

The present process according to the invention can correspondingly also be applied to a process for preparing methacrylic acid by heterogeneously catalyzed partial oxidation of at least one C₄ hydrocarbon precursor compound (e.g. isobutene or isobutane).

### EXAMPLES AND COMPARATIVE EXAMPLES

A) Preparation of an Annular Unsupported Catalyst UCM for the First Reaction Stage (Propene→Acrolein) of the Main Reaction Having the Stoichiometry
[Bi₂W₂O₉.2WO₃]_{0.5}[Mo₁₂Co_{5.5}Fe_{2.94}Si_{1.59}K_{0.06}Oₓ]₁
1. Preparation of a Starting Composition 1

209.3 kg of tungstic acid (72.94% by weight of W) were stirred in portions into 775 kg of an aqueous bismuth nitrate solution in nitric acid (11.2% by weight of Bi; free nitric acid from 3 to 5% by weight; mass density: from 1.22 to 1.27 g/ml) at 25° C. The resulting aqueous mixture was subsequently stirred at 25° C. for a further 2 h and subsequently spray-dried.

The spray-drying was effected in a rotating disk spray tower in countercurrent at a gas inlet temperature of 300±10° C. and a gas outlet temperature of 100±10° C. The resulting spray powder (particle size a substantially uniform 30 μm) which had an ignition loss of 12% by weight (ignite at 600° C. under air for 3 h) was subsequently converted to a paste in a kneader using 16.8% by weight (based on the powder) of water and extruded by means of an extruder (rotational moment: ≦50 Nm) to extrudates of diameter 6 mm. These were cut into sections of 6 cm, dried under air on a 3-zone belt dryer at a residence time of 120 min at temperatures of 90-95° C. (zone 1) and 125° C. (zone 2) and 125° C. (zone 3), and then thermally treated at a temperature in the range from 780 to 810° C. (calcined; in a rotary tube oven flowed through by air (capacity 1.54 m³, 200 m³ (STP) of air/h)). When precisely adjusting the calcination temperature, it is essential that it has to be directed to the desired phase composition of the calcination product. The desired phases are WO₃ (monoclinic) and Bi₂W₂O₉; the presence of γ-Bi₂WO₆ (Russellite) is undesired. Therefore, should the compound γ-Bi₂WO₆ still be detectable by a reflection in the X-ray powder diffractogram after the calcination at a reflection angle of 2⊖=28.4° (Cuka radiation), the preparation has to be repeated and the calcination temperature increased within the temperature range specified or the residence time increased at constant calcination temperature, until the disappearance of the reflection is achieved. The preformed calcined mixed oxide obtained in this way was ground so that the X₅₀ value (cf. Ullmann's Encyclopedia of Industrial Chemistry, 6^{th} Edition (1998) Electronic Release, Chapter 3.1.4 or DIN 66141) of the resulting particle size was 5 mm. The ground material was then mixed with 1% by weight (based on the ground material) of finely divided SiO₂ from Degussa of the Sipernat® type (bulk density 150 g/l; X₅₀ value of the SiO₂ particles was 10 μm, the BET surface area was 100 m²/g).

2. Preparation of a Starting Composition 2

A solution A was prepared by dissolving 213 kg of ammonium heptamolybdate tetrahydrate (81.5% by weight of MoO₃) at 60° C. with stirring in 600 l of water and the resulting solution was admixed while maintaining the 60° C. and stirring with 0.97 kg of an aqueous potassium hydroxide solution (46.8% by weight of KOH) at 20° C.

A solution B was prepared by introducing 116.25 kg of an aqueous iron(III) nitrate solution (14.2% by weight of Fe) at 60° C. into 262.9 kg of an aqueous cobalt(II) nitrate solution (12.4% by weight of Co). Subsequently, while maintaining the 60° C., solution B was continuously pumped into the initially charged solution A over a period of 30 minutes. Subsequently, the mixture was stirred at 60° C. for 15 minutes. 19.16 kg of a Ludox silica gel from Dupont (46.80% by weight of SiO₂, density: from 1.36 to 1.42 g/ml, pH from 8.5 to 9.5, max. alkali content 0.5% by weight) were then added to the resulting aqueous mixture, and the mixture was stirred afterward at 60° C. for a further 15 minutes.

Subsequently, the mixture was spray-dried in countercurrent in a rotating disk spray tower (gas inlet temperature: 400±10° C., gas outlet temperature: 140±5° C.). The resulting spray powder had an ignition loss of approx. 30% by weight (ignite under air at 600° C. for 3 h) and a substantially uniform particle size of 30 μm.

Preparation of the Multimetal Oxide Active Composition

The starting composition 1 was mixed homogeneously with the starting composition 2 in the amounts required for a multimetal oxide active composition of the stoichiometry
[Bi₂W₂O₉.2WO₃]_{0.5}[Mo₁₂Co_{5.5}Fe_{2.94}Si_{1.59}K_{0.08}Oₓ]₁
in a mixer having bladed heads. Based on the aforementioned overall composition, an additional 1% by weight of finely divided graphite from Timcal AG (San Antonio, US) of the TIMREX P44 type (sieve analysis: min. 50% by weight ≦24 μm, max. 10% by weight ≧24 μm and ≦48 μm, max. 5% by weight >48 μm, BET surface area: from 6 to 13 m²/g) were mixed in homogeneously. The resulting mixture was then conveyed in a compactor (from Hosokawa Bepex GmbH, D-74211 Leingarten) of the K200/100 compactor type having concave, fluted smooth rolls (gap width: 2.8 mm, sieve width: 1.0 mm, lower particle size sieve width: 400 μm, target compressive force: 60 kN, screw rotation rate: from 65 to 70 revolutions per minute). The resulting compactate had a hardness of 10 N and a substantially uniform particle size of from 400 μm to 1 mm.

The compactate was subsequently mixed with, based on its weight, a further 2% by weight of the same graphite and subsequently compressed in a Kilian rotary tableting press of the R x 73 type from Kilian, D-50735 Cologne, under a nitrogen atmosphere to give annular shaped unsupported catalyst precursor bodies of geometry (external diameter×length×internal diameter) 5 mm×3 mm×2 mm and having a side crushing strength of 19 N±3 N.

In this document, side crushing strength refers to the crushing strength when the annular shaped unsupported catalyst precursor body is compressed at right angles to the cylinder surface (i.e. parallel to the surface of the ring orifice).

All side crushing strengths in this document relate to a determination by means of a material testing machine from Zwick GmbH & Co. (D-89079 Ulm) of the Z 2.5/TS1S type. This material testing machine is designed for quasistatic stress having an uninterrupted, stationary, dynamic or varying profile. It is suitable for tensile, compressive and bending tests. The installed force transducer of the KAF-TC type from A.S.T. (D-01307 Dresden) having the manufacturer number 03-2038 was calibrated in accordance with DIN EN ISO 7500-1 and could be used for the 1-500 N measurement range (relative measurement uncertainty: ±0.2%).

The measurements were carried out with the following parameters:
Initial force: 0.5 N.
Rate of initial force: 10 mm/min.
Testing rate: 1.6 mm/min.

The upper die was initially lowered slowly down to just above the cylinder surface of the annular shaped unsupported catalyst precursor body. The upper die was then stopped, in order subsequently to be lowered at the distinctly slower testing rate with the minimum initial force required for further lowering.

The initial force at which the shaped unsupported catalyst precursor body exhibits crack formation is the side crushing strength (SCS).

For the final thermal treatment, in each case 1000 g of the shaped unsupported catalyst precursor bodies were heated in a muffle furnace flowed through by air (capacity 60 l, 1 l/h of air per gram of shaped unsupported catalyst precursor body) initially from room temperature (25° C.) to 190° C. at a heating rate of 180° C./h. This temperature was maintained for 1 h and then increased to 210° C. at a heating rate of 60° C./h. The temperature of 210° C. was in turn maintained over 1 h before it was increased to 230° C. at a heating rate of 60° C./h. This temperature was likewise maintained for 1 h before it was increased to 265° C., again at a heating rate of 60° C./h. The temperature of 265° C. was subsequently likewise maintained over 1 h. Afterward, the furnace was initially cooled to room temperature and the decomposition phase thus substantially completed. The furnace was then heated to 465° C. at a heating rate of 180° C./h and this calcination temperature maintained over 4 h.

Annular unsupported catalysts UCM were obtained from the annular shaped unsupported catalyst precursor bodies.

The specific surface area S, the total pore volume V, the pore diameter d^{max} which makes the greatest contribution to the total pore volume, and the percentages of those pore diameters in the total pore volume whose diameter is >0.1 and ≦1 μm for the resulting annular unsupported catalysts UCM were as follows:
S=7.6 cm²/g.
V=0.27 cm³/g.
d^{max}[μm]=0.6.
V^{0.1}₁₋%=79.

In addition, the ratio R of apparent mass density to true mass density p (as defined in EP-A 1340538) was 0.66.

On the industrial scale, the same annular catalyst was prepared by means of a belt calcining apparatus by thermal treatment as described in example 1 of DE-A 10046957 (however, the bed height in the decomposition (chambers 1 to 4) was advantageously 44 mm at a residence time per chamber of 1.46 h and in the calcination (chambers 5 to 8) it was advantageously 130 mm at a residence time of 4.67 h); the chambers had a surface area (at a uniform chamber length of 1.40 m) of 1.29 m² (decomposition) and 1.40 m² (calcination) and were flowed through from below through the coarse-mesh belt by an air supply of 75 m³ (STP)/h which was aspirated by means of rotating ventilators. Within the chambers, the temporal and local deviation of the temperature from the target value was always ≦2° C. Otherwise, the procedure was as described in example 1 of DE-A 10046957.

Alternatively, it was also possible to use for the first reaction stage an annular unsupported catalyst of the same geometry and preparation method, but having the stoichiometry [Bi₂W_{1.91}O₉.1.91WO₃]_{0.5}[Mo_{12.7}Co_{5.79}Fe_{3.22}Si_{1.66}K_{0.08}Oₓ]₁.

B) Preparation of an Annular Coated Catalyst CCM1 for the Second Reaction Stage (Acrolein Acrylic Acid) of the Main Reaction Having the Stoichiometry Mo₁₂V₃W_{1.2}Cu_{2.4}Oₓ of the active composition

1. General Description of the Rotary Tube Furnace Used for Calcination

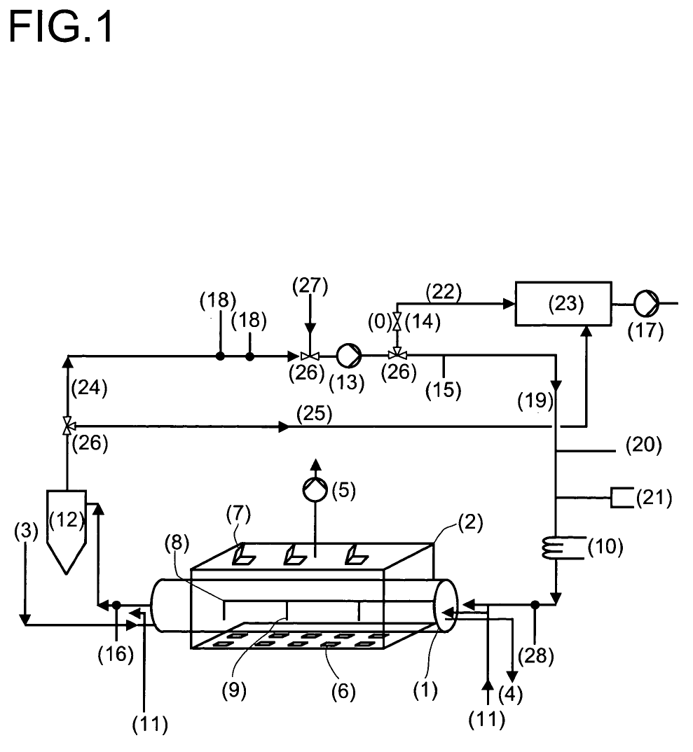

A schematic diagram of the rotary tube furnace is shown by the FIG. 1 appended to this document. The reference numerals which follow relate to this FIG. 1.

The central element of the rotary tube furnace is the rotary tube (1). It is 4000 mm long and has an internal diameter of 700 mm. It is manufactured from 1.4893 stainless steel and has a wall thickness of 10 mm.

On the interior wall of the rotary tube furnace are mounted lifting lances which have a height of 5 cm and a length of 23.5 cm. They primarily serve the purpose of lifting the material to be thermally treated in the rotary tube furnace, thus mixing it.

At one and the same height of the rotary tube furnace are mounted in each case four lifting lances (a quadruple) equidistantly around the circumference (separation in each case 90°). Along the rotary tube furnace are disposed eight such quadruples (each 23.5 cm apart). The lifting lances of two adjacent quadruples are offset relative to one another on the circumference. At the start and at the end of the rotary tube furnace (first and last 23.5 cm) there are no lifting lances.

The rotary tube rotates freely in a cuboid (2) which has four electrically heated (resistance heating) heating zones which are successive in the length of the rotary tube and are of equal length, each of which encloses the circumference of the rotary tube furnace. Each of the heating zones can heat the appropriate rotary tube section to temperatures between room temperature and 850° C. The maximum heating output of each heating zone is 30 kW. The distance between electrical heating zone and rotary tube exterior surface is about 10 cm. At the start and at the end, the rotary tube projects approx. 30 cm beyond the cuboid.

The rotation rate may be variably adjusted between 0 and 3 revolutions per minute. The rotary tube can be rotated either to the left or to the right. In the case of rotation to the right, the material remains in the rotary tube; in the case of rotation to the left, the material is conveyed from inlet (3) to outlet (4). The inclination angle of the rotary tube to the horizontal may be variably adjusted between 0° and 2°. In batchwise operation, it is in fact 0°. In continuous operation, the lowermost point of the rotary tube is at the material outlet. The rotary tube may be rapidly cooled by switching off the electrical heating zones and switching on a ventilator (5). This aspirates ambient air through holes (6) in the lower base of the cuboid, and conveys it through three flaps (7) having variably adjustable opening in the lid.

The material inlet is controlled via a rotary star feeder (mass control). The material output is, as already mentioned, controlled via the rotation direction of the rotary tube.

In the case of batchwise operation of the rotary tube, an amount of material of from 250 to 500 kg may be thermally treated. The amount is normally disposed exclusively in the heated section of the rotary tube.

From a lance (8) lying on the central axis of the rotary tube, a total of three thermo-elements (9) lead vertically into the material at intervals of 800 mm. They enable the determination of the temperature of the material. In this document, temperature of the material refers to the arithmetic mean of the three thermoelement temperatures. According to the invention, the maximum deviation of two measured temperatures within the material in the rotary tube is appropriately less than 30° C., preferably less than 20° C., more preferably less than 10° C. and most preferably less than 5 or 3° C.

Gas streams may be conducted through the rotary tube, by means of which the calcination atmosphere or generally the atmosphere of the thermal treatment of the material can be adjusted.

The heater (10) offers the possibility of heating the gas stream conducted into the rotary tube to the desired temperature before its entry into the rotary tube (for example to the temperature desired in the rotary tube for the material). The maximum output of the heater is 1×50 kW+1×30 kW. In principle, the heater (10) may be, for example, an indirect heat exchanger. Such a heater may in principle also be used as a cooler. However, it is generally an electrical heater in which the gas stream is conducted over metal wires heated using electricity (appropriately a 97D/80 CSN flow heater from C. Schniewindt K G, 58805 Neuerade, Germany).

In principle, the rotary tube apparatus provides the possibility of partly or fully recycling the gas stream conducted through the rotary tube. The recycle line required for this purpose is connected to the rotary tube in a mobile manner at the rotary tube inlet and at the rotary tube outlet using ball bearings or using graphite pressure seals. These connections are flushed with inert gas (e.g. nitrogen) (barrier gas). The two flush streams (11) supplement the gas stream conducted through the rotary tube at the inlet into the rotary tube and at the outlet from the rotary tube. Appropriately, the rotary tube narrows at its start and at its end and projects into the tube of the recycle line leading to and away from it respectively.

Downstream of the outlet of the gas stream conducted through the rotary tube is disposed a cyclone (12) to remove solid particles entrained with the gas stream (the centrifugal separator separates solid particles suspended in the gas phase by interaction of centrifugal force and gravity; the centrifugal force of the gas stream rotating as a spiral accelerates the sedimentation of the suspended particles).

The conveying of the cycle gas stream (24) (the gas circulation) is effected by means of a cycle gas compressor (13) (ventilator) which aspirates in the direction of the cyclone and forces in the other direction. Directly downstream of the cycle gas compressor, the gas pressure is generally above one atmosphere. Downstream of the cycle gas compressor is disposed a cycle gas outlet (cycle gas may be discharged via a regulating valve (14)). A diaphragm disposed downstream of the outlet (cross-sectional reduction by about a factor of 3, pressure reducer) (15) eases the discharge.

The pressure downstream of the rotary tube outlet can be controlled via the regulating valve. This is effected in combination with a pressure sensor (16) mounted downstream of the rotary tube outlet, the offgas compressor (17) (ventilator) which aspirates toward the regulating valve, the cycle gas compressor (13) and the fresh gas feed. Relative to the external pressure, the pressure (directly) downstream of the rotary tube outlet may be set, for example, to up to +1.0 mbar higher and, for example, up to −1.2 mbar lower. In other words, the pressure of the gas stream flowing through the rotary tube may be below the ambient pressure of the rotary tube when it leaves the rotary tube.

When the intention is not to at least partly recycle the gas stream conducted through the rotary tube, the connection between cyclone (12) and cycle gas compressor (13) is made by the three-way valve principle (26) and the gas stream is conducted directly into the offgas cleaning apparatus (23). The connection to the offgas cleaning apparatus disposed downstream of the cycle gas compressor is in this case likewise made by the three-way valve principle. When the gas stream consists substantially of air, it is in this case aspirated (27) via the cycle gas compressor (13). The connection to the cyclone is made by the three-way valve principle. In this case, the gas stream is preferably sucked through the rotary tube, so that the internal rotary tube pressure is less than the ambient pressure.

In the case of continuous operation of the rotary tube furnace apparatus, the pressure downstream of the rotary tube outlet is advantageously set −0.2 mbar below the external pressure. In the case of batchwise operation of the rotary tube apparatus, the pressure downstream of the rotary tube outlet is advantageously set −0.8 mbar below the external pressure. The slightly reduced pressure serves the purpose of preventing contamination of the ambient air with gas mixture from the rotary tube furnace.

Between the cycle gas compressor and the cyclone are disposed sensors (18) which determine, for example, the ammonia content and the oxygen content in the cycle gas. The ammonia sensor preferably operates by an optical measurement principle (the absorption of light of a certain wavelength correlates proportionally to the ammonia content of the gas) and is appropriately an MCS 100 instrument from Perkin & Elmer. The oxygen sensor is based on the paramagnetic properties of oxygen and is appropriately an Oxymat from Siemens of the Oxymat MAT SF 7 MB1010-2CA01-1AA1-Z type.

Between the diaphragm (15) and the heater (10), gases such as air, nitrogen, ammonia or other gases may be metered into the cycle gas fraction (19) which is actually to be recirculated. Frequently, a base load of nitrogen is metered in (20). A separate nitrogen/air splitter (21) may be used to react to the measurement of the oxygen sensor.

The discharged cycle gas fraction (22) (offgas) frequently comprises gases such as. NOₓ, acetic acid, NH₃, etc. which are not entirely safe, which is why they are normally removed in an offgas cleaning apparatus (23).

To this end, the offgas is generally initially conducted through a washing column (essentially a column free of internals which comprises a separating structured packing upstream of its outlet; the offgas and aqueous sprayed mist are conducted in cocurrent and in countercurrent (2 spray nozzles having opposite spray direction)).

Exiting the washing column, the offgas is conducted into an apparatus which comprises a fine dust filter (generally a series of bag filters) from whose interior the penetrant offgas is discharged. Finally, incineration is effected in a muffle furnace.

A sensor (28) from KURZ Instruments, Inc., Monterey (USA) of the 455 Jr model is used to measure and control the flow rate of the gas stream which is fed to the rotary tube and is different to the barrier gas (measurement principle: thermal-convective mass flow measurement using an isothermal anemometer).

In the case of continuous operation, material and gas phase are conducted through the rotary tube furnace in countercurrent.

In connection with this example, nitrogen always means nitrogen having a purity of >99% by volume.

2. Preparation of a Precursor Composition for the Purpose of Obtaining a Multielement Oxide Composition of the Stoichiometry Mo₁₂V₃W_{1.2}Cu_{2.4}Oₓ

16.3 kg of copper(II) acetate hydrate (content: 40.0% by weight of CuO) were dissolved with stirring in 274 l of water at a temperature of 25° C. A clear solution 1 was obtained.

Spatially separately therefrom, 614 l of water were heated to 40° C. and 73 kg of ammonium heptamolybdate tetrahydrate (81.5% by weight of MoO₃) were stirred in while maintaining the 40° C. The mixture was then heated with stirring to 90° C. within 30 min and, while retaining this temperature, successively and in the sequence mentioned, 12.1 kg of ammonium metavanadate and 10.7 kg of ammonium paratungstate heptahydrate (88.9% by weight of WO₃) were stirred in (instead of the 12.1 kg of ammonium metavanadate, it is alternatively also possible to use 11.3 kg and otherwise proceed as described; in this case, the resulting active composition alternative is one of the stoichiometry Mo₁₂V_{2.8}W_{1.2}Cu_{2.4} Oₓ). A clear solution 2 was obtained.

Solution 2 was cooled to 80° C. and subsequently solution 1 was stirred into solution 2. The resulting mixture was admixed with 130 l of a 25% by weight aqueous NH₃ solution which had a temperature of 25° C. With stirring, a clear solution was formed which briefly had a temperature of 65° C. and a pH of 8.5. To this were once again added 20 l of water at a temperature of 25° C. Afterward, the temperature of the resulting solution rose again to 80° C. and the solution was then spray-dried using an S-50-N/R spray dryer from Niro-Atomizer (Copenhagen) (gas inlet temperature: 350° C., gas outlet temperature: 110° C.). The spray powder had a particle diameter of from 2 to 50 μm. 60 kg of thus obtained spray powder were metered into a VM 160 kneader (Sigma blades) from AMK (Aachener Misch-und Knetmaschinen Fabrik) and kneaded with the addition of 5.5 l of acetic acid (≈100% by weight, glacial acetic acid) and 5.2 l of water (rotation rate of the screw: 20 rpm). After a kneading time of from 4 to 5 minutes, a further 6.5 l of water were added and the kneading process was continued until 30 minutes had elapsed (kneading temperature from approx. 40 to 50° C.). Afterward, the kneaded material was emptied into an extruder and shaped by means of the extruder (from Bonnot Company (Ohio), model: G 103-10/D7A-572K (6″ Extruder W Packer)) to extrudates (length: 1-10 cm; diameter 6 mm). On the belt dryer, the extrudates were dried at a temperature of 120° C. (material temperature) for 1 h. The dried extrudates formed the precursor composition to be treated thermally.

3. Preparation of the Catalytic Active Composition by Thermal Treatment of the Precursor Composition (Calcination of the Precursor Composition) in a Rotary Tube Furnace Apparatus

The thermal treatment was carried out in the rotary tube furnace described under “B) 1.” according to FIG. 1 and under the following conditions:
the thermal treatment was effected batchwise using a material amount of 300 kg which had been prepared as described in “B) 2.”;
the inclination angle of the rotary tube to the horizontal was ≈0°;
the rotary tube rotated to the right at 1.5 revolutions/min;
over the entire thermal treatment, a gas stream of 205 m³ (STP)/h was conducted through the rotary tube and (after displacement of the air originally present) had the following composition and was supplemented at its outlet from the rotary tube by a further 25 m³ (STP)/h of barrier gas nitrogen:
80 m³ (STP)/h composed of baseload nitrogen (20) and gases released in the rotary tube,
25 m³ (STP)/h of barrier gas nitrogen (11),
30 m³ (STP)/h air (splitter (21)); and
70 m³ (STP)/h of recirculated cycle gas (19).

The barrier gas nitrogen was fed at a temperature of 25° C. The mixture of the other gas streams, coming from the heater, was in each case conducted into the rotary tube at the temperature that the material had in each case in the rotary tube.
within 10 h, the material temperature of 25° C. was heated in a substantially linear manner to 300° C.;
subsequently, the material temperature was heated within 2 h in a substantially linear manner to 360° C.;
subsequently, the material temperature was reduced to 350° C. in a substantially linear manner within 7 h;
then the material temperature was increased to 420° C. in a substantially linear manner within 2 h and this material temperature was held over 30 min;
then the 30 m³ (STP)/h of air in the gas stream conducted through the rotary tube were replaced by a corresponding increase in the baseload nitrogen (which ended the procedure of the actual thermal treatment), the heating of the rotary tube was switched off and the material was cooled by switching on the rapid cooling of the rotary tube by aspirating ambient air to a temperature below 100° C. within 2 h and finally to ambient temperature; the gas stream was fed to the rotary tube at a temperature of 25° C.;
over the entire thermal treatment, the pressure (directly) downstream of the rotary tube outlet of the gas stream was 0.2 mbar below the external pressure.

The oxygen content of the gas atmosphere in the rotary tube furnace in all phases of the thermal treatment was 2.99% by volume. Arithmetically averaged over the entire duration of the reductive thermal treatment, the ammonia concentration of the gas atmosphere in the rotary tube furnace was 4% by volume.

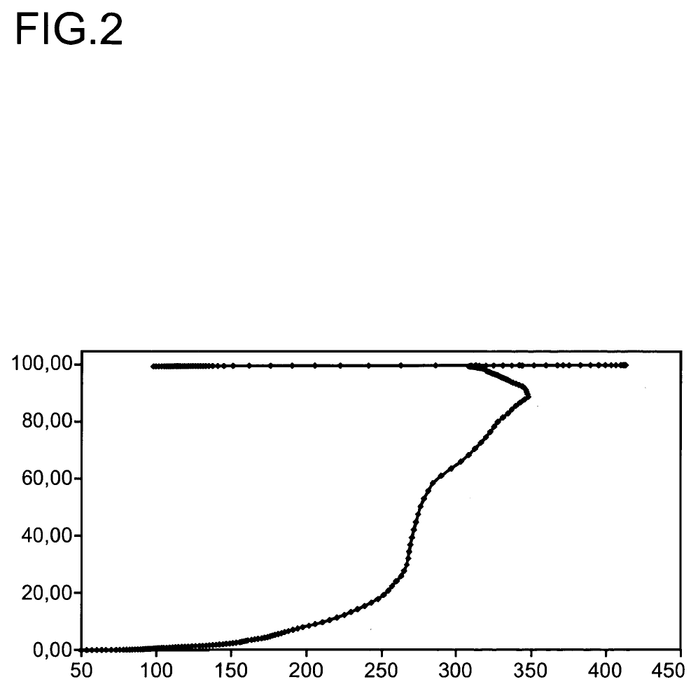

FIG. 2 shows the amount of ammonia released from the precursor composition as a function of the material temperature in ° C. as a percentage of the total amount of ammonia released from the precursor composition within the overall course of the thermal treatment.

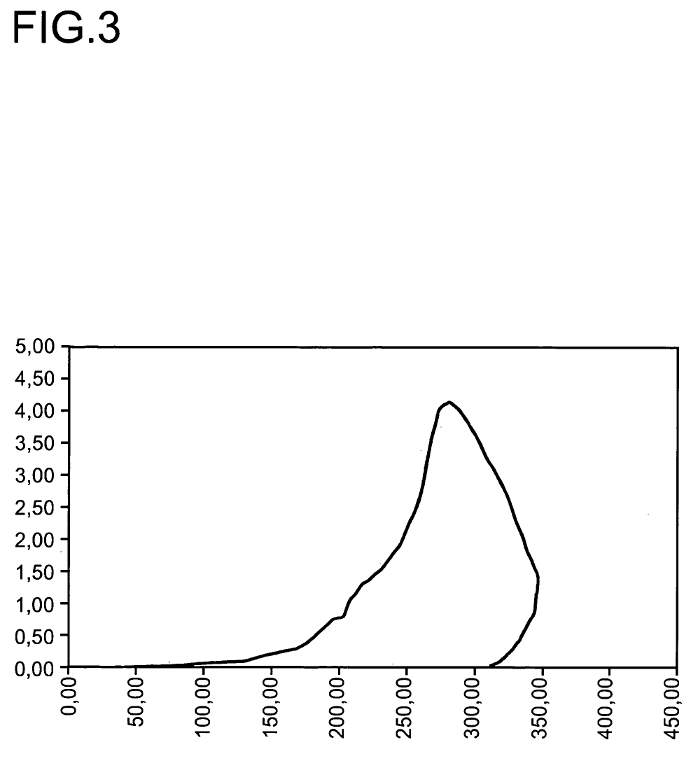

FIG. 3 shows the ammonia concentration of the atmosphere in % by volume in which the thermal treatment was effected as a function of the material temperature in ° C. during the thermal treatment.

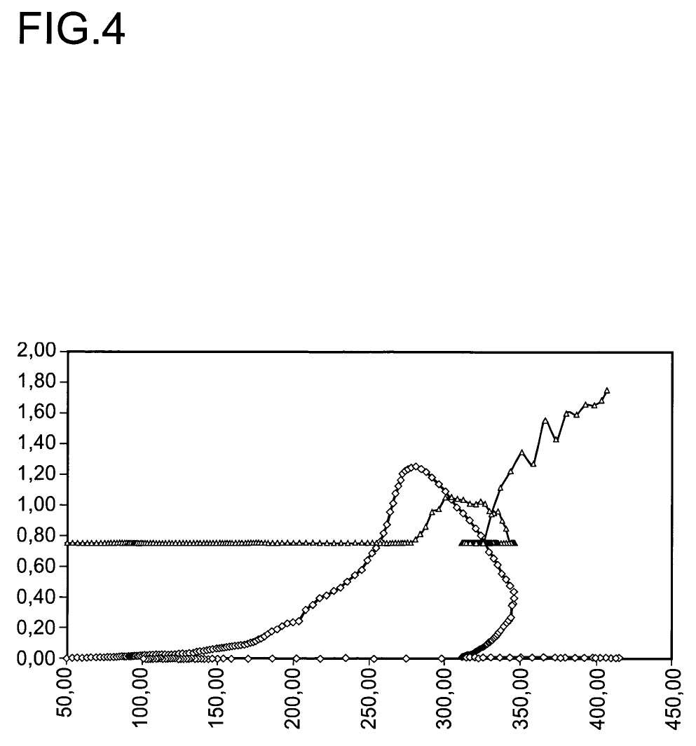

FIG. 4 shows, as a function of the material temperature, the molar amounts of molecular oxygen and of ammonia which were conducted into the rotary tube per kg of precursor composition and hour over the thermal treatment with the gas stream.

4. Shaping of the Multimetal Oxide Active Composition

The catalytically active material obtained under “B)3.” was ground by means of a BQ500 Biplex crossflow classifying mill (Hosokawa-Alpine Augsburg) to give a fine powder of which 50% of the powder particles passed through a sieve of mesh width from 1 to 10 μm and whose proportion of particles of longest dimension above 50 μm was less than 1%.

The shaping was then effected as follows:

70 kg of annular support bodies (external diameter 7.1 mm, length 3.2 mm, internal diameter 4.0 mm; type C220 steatite from CeramTec having a surface roughness R_{z} of 45 μm and a total pore volume based on the volume of the support body of ≦1% by volume; cf. DE-A 2135620) were charged into a coating tank (inclination angle 90°; Hicoater from Lödige, Germany) of capacity 200 l. Subsequently, the coating tank was set into rotation at 16 rpm. A nozzle was used to spray from 3.8 to 4.2 liters of an aqueous solution composed of 75% by weight of water and 25% by weight of glycerol onto the support bodies within 25 min. At the same time, 18.1 kg of the ground multimetal oxide active composition (whose specific surface area was 13.8 m²/g) were metered in continuously within the same period via an agitated channel outside the spray cone of the atomizer nozzle. During the coating, the powder supplied was fully taken up on the surface of the support body; no agglomeration of the finely divided oxidic active composition was observed. On completion of addition of active composition powder and water, hot air (approx. 400 m³/h) at 100° C. (alternatively from 80 to 120° C.) was blown into the coating tank at a rotation rate of 2 rpm for 40 min (alternatively from 15 to 60 min). Annular coated catalysts CCM1 were obtained whose proportion of oxidic active composition based on the overall composition was 20% by weight. The coating thickness, viewed both over the surface of one support body and over the surface of different support bodies, was 170±50 μm.

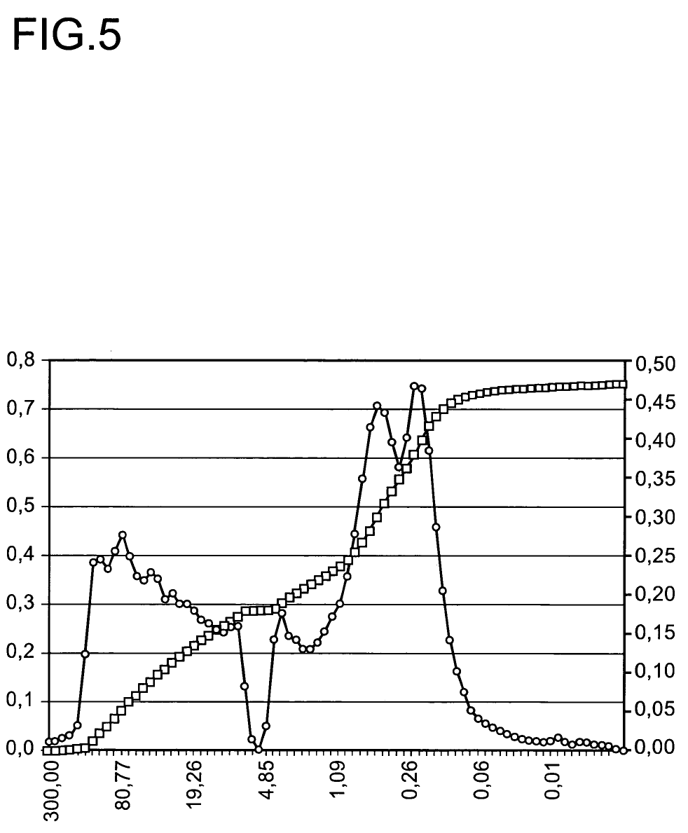

FIG. 5 also shows the pore distribution of the ground active composition powder before it is shaped. On the abscissa is plotted the pore diameter in μm (logarithmic scale).

On the right ordinate is plotted the logarithm of the differential contribution in ml/g of the particular pore diameter to the total pore volume (O curve). The maximum indicates the pore diameter having the greatest contribution to the total pore volume. On the left ordinate is plotted in ml/g the integral over the individual contributions of the individual pore diameters to the total pore volume (l curve). The end point is the total pore volume (unless stated otherwise, all data in this document on determinations of total pore volumes and on diameter distributions to these total pore volumes relate to determinations by the method of mercury porosimetry using the Auto Pore 9220 instrument from Micromeritics GmbH, 4040 Neuss, Germany (bandwidth from 30 Å to 0.3 mm); all data in this document on determinations of specific surface areas or of micropore volumes relate to determinations to DIN 66131 (determination of the specific surface area of solids by Brunauer-Emmet-Teller (BET) gas adsorption (N₂))).

FIG. 6 shows, for the active composition powder before it is shaped, in ml/g (ordinate), the individual contributions of the individual pore diameters (abscissa, in angström, logarithmic scale) in the micropore range to the total pore volume.

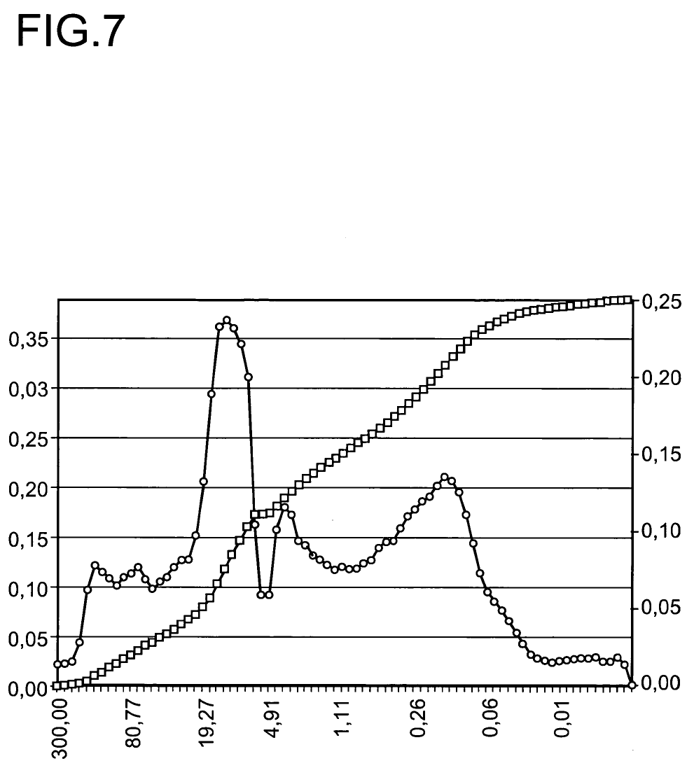

FIG. 7 shows the same as FIG. 5, but for multimetal oxide active composition subsequently removed from the annular coated catalyst by mechanical scraping (its specific surface area was 12.9 m²/g).

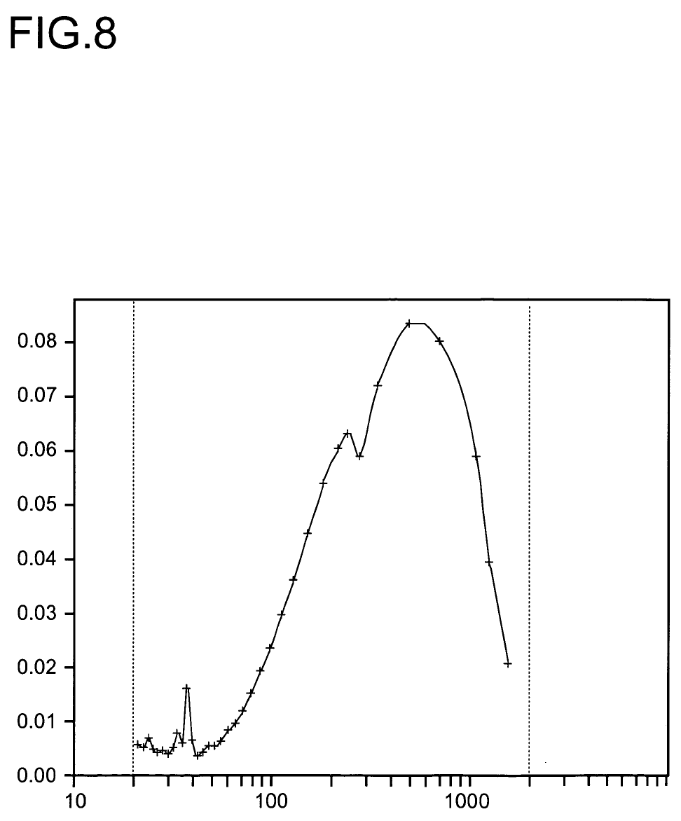

FIG. 8 shows the same as FIG. 6, but for multimetal oxide active composition subsequently removed from the annular coated catalyst by mechanical scraping.

C) Preparation of an Annular Coated Catalyst CCP1 for the Postreaction Stage Having the Stoichiometry Mo₁₂V₃W_{1.2}Cu_{2.4}Pd_{0.03}O ₓ of the active composition

The preparation was as for the preparation of CCM1, but with the difference that after solution 1 had been stirred into solution 2, 225 g of solid Pd(NO₃)₂ hydrate (manufacturer: Sigma-Aldrich) were stirred into the resulting mixture before the aqueous NH₃ solution was added.

D) Preparation of an Annular Coated Catalyst CCP2 for the Postreaction Stage Having the Stoichiometry Mo₁₂V₃W_{1.2}Cu_{2.4}Fe_{1.2}Oₓ of the active composition

The preparation was as for the preparation of CCM1, but with the difference that after solution 1 had been stirred into solution 2, 16.7 kg of solid Fe(NO₃)₂.9H₂O (manufacturer: Riedel-de Haen, 97%) were stirred into the resulting mixture before the aqueous NH₃ solution was added.

E) Preparation of an Annular Coated Catalyst CCP3 for the Postreaction Stage Having the Stoichiometry Mo₁₂V₃W_{1.2}Cu_{4.8}Oₓ of the active composition

The preparation was as for the preparation of CCM1, but with the difference that solution 1, instead of 16.3 kg of copper(II) acetate hydrate, comprised 32.6 kg of the same copper(II) acetate hydrate.

F) Preparation of an Annular Coated Catalyst CCP4 for the Postreaction Stage Having the Stoichiometry (Mo₁₂V₃W_{1.2}Cu_{1.9}Oₓ)(Fe₁Sb₂O₅)_{0.5} as the active composition

The preparation was as for the preparation of CCM1, but with the following differences:
instead of 16.3 kg of copper(II) acetate hydrate, solution 1 comprised only 12.9 kg of the same copper(II) acetate hydrate;
however, the resulting spray powder of the stoichiometry Mo₁₂V₃W_{1.2}Cu_{1.9} was not further processed as such in the kneader, but rather mixed homogeneously with finely divided FeSb₂O₆ in a weight ratio of 15:1;
the finely divided FeSb₂O₆ had been obtained as follows:

838.36 g of antimony trioxide (Sb₂O₃, senarmontite) was added in finely divided form to 3545 g of water and, after addition of 728.47 g of a 30% by weight solution of H₂O₂ in water, heated to reflux at atmospheric pressure over 5 h, and a solution 1 was thus obtained.

500 g of iron(II) acetate (31.5% by weight of Fe, supplier: ABCR) were dissolved in 3545 g of water to give a solution 2. The solution 2 at room temperature was subsequently added within 30 min to the boiling solution 1. The temperature of the resulting mixture was 70° C. This ochre-colored suspension was stirred at 80° C. over a further 3 h and finally spray-dried using the Niro-Atomizer (Copenhagen) spray dryer already described (gas inlet temperature: 350° C., gas outlet temperature: 110° C.). The resulting spray powder had a particle diameter of from 2 to 50 μm.

G) Preparation of an Annular Coated Catalyst CCP5 for the Postreaction Stage Having the Stoichiometry Mo₁V_{0.29}Te_{0.14}Nb_{0.13}Oₓ of the Active Composition

87.61 g of ammonium metavanadate (78.55% by weight of V₂O₅, from G.f.E., Nuremberg, Germany) were dissolved at 80° C. in 3040 ml of water in a glass three-neck flask equipped with stirrer, thermometer, reflux condenser and heating. This gave a clear yellowish solution. This solution was cooled to 60° C. and then, successively in the sequence specified, while maintaining the 60° C., first 117.03 g of telluric acid (99% by weight of H₆TeO₆, from Aldrich) and then 400 g of ammonium heptamolybdate tetrahydrate (82.52% by weight of MoO₃, from Starck, Goslar, Germany) were stirred in. The resulting deep red solution was cooled to 30° C. and a solution A was obtained in this way.

In a beaker, 112.67 g of ammonium niobium oxalate (20.8% by weight of Nb, from Starck, Goslar, Germany) in 500 ml of water were dissolved separately at 60° C. to give a solution B. Solution B was likewise cooled to 30° C. and combined at this temperature with solution A by stirring solution B into solution A. The stirring-in was effected continuously within a period of 5 minutes. This gave an orange-colored aqueous suspension.

This suspension was subsequently spray-dried (Tᵣₑₛₑᵥₒᵢᵣ=30° C., Tⁱⁿ=320° C., Tₒᵤₜ=110° C., drying time: approx. 1.5 h, spray tower from Niro, Germany, of the Atomizer type). The sprayed material was likewise orange.

1% by weight of finely divided graphite (sieve analysis: min. 50% by weight ≦24 μm, max. 10% by weight >24 μm and ≦48 μm, max. 5% by weight >48 μm, BET surface area: from 6 to 13 m²/g) was mixed into the sprayed material.

The resulting mixture was compacted (compressed) to hollow cylinders (rings) of geometry 16 mm×2.5 mm×8 mm (external diameter×height×internal diameter) in such a way that the resulting side crushing strength of the rings was approx. 10 N.

200 g of these rings were calcined in two portions each of 100 g successively in a rotary sphere furnace according to FIG. 1 of DE-A 10122027. To this end, the rotary sphere furnace contents were heated from 25° C. to 275° C. within 27.5 min with a linear heating ramp under an air stream of 50 l (STP)/h, and kept at this temperature for 1 h while maintaining the air stream. Subsequently, the furnace was heated from 275° C. to 600° C. with a linear heating ramp within 32.5 min, in the course of which the air stream was replaced by a nitrogen stream of 50 l (STP)/h. The 600° C. and the nitrogen stream were maintained for 2 h and the entire furnace was subsequently left to cool to 25° C. while maintaining the nitrogen stream. This resulted in black rings of the composition Mo_{1.0}V_{0.33}Te_{0.19}Nb_{0.11}Oₓ(stoichiometry: Mo_{1.0}V_{0.33}Te_{0.22}Nb_{0.11}Oₓ).

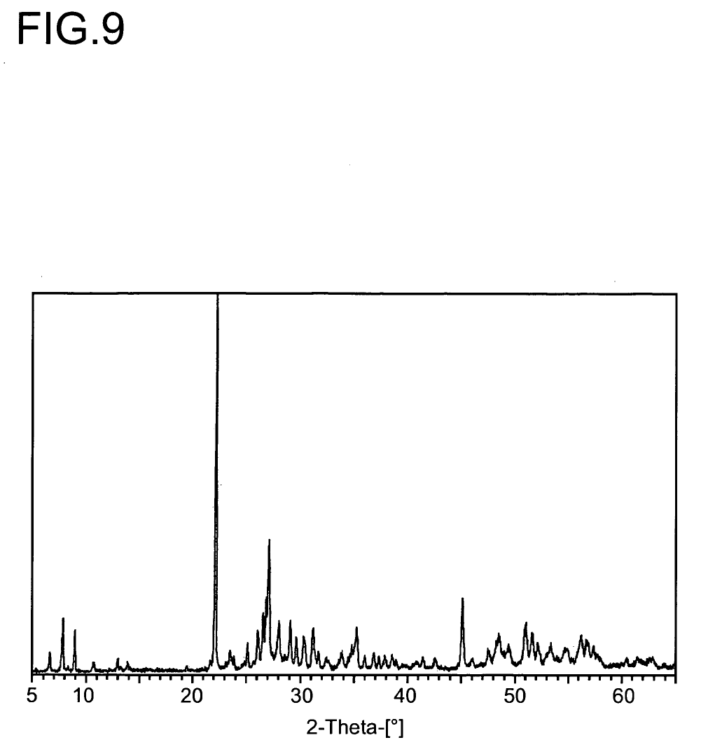

In a Retsch mill, the rings were ground dry to a particle size of ≦100 μm. 100 g of the ground material were stirred under reflux at 70° C. in 1000 ml of a 10% by weight aqueous HNO₃ solution over 7 h, and the solid was filtered out of the resulting slurry and washed with water to free it of nitrate. The filtercake was dried in a muffle furnace at 110° C. under air overnight. The resulting active composition had the composition Mo_{1.0}V_{0.29}Te_{0.14}Nb_{0.13}Oₓ. Its X-ray diffractogram (cf. FIG. 9) revealed pure i phase having R=0.74. It contained no reflection having the peak location 2⊖=50.0±0.3°.

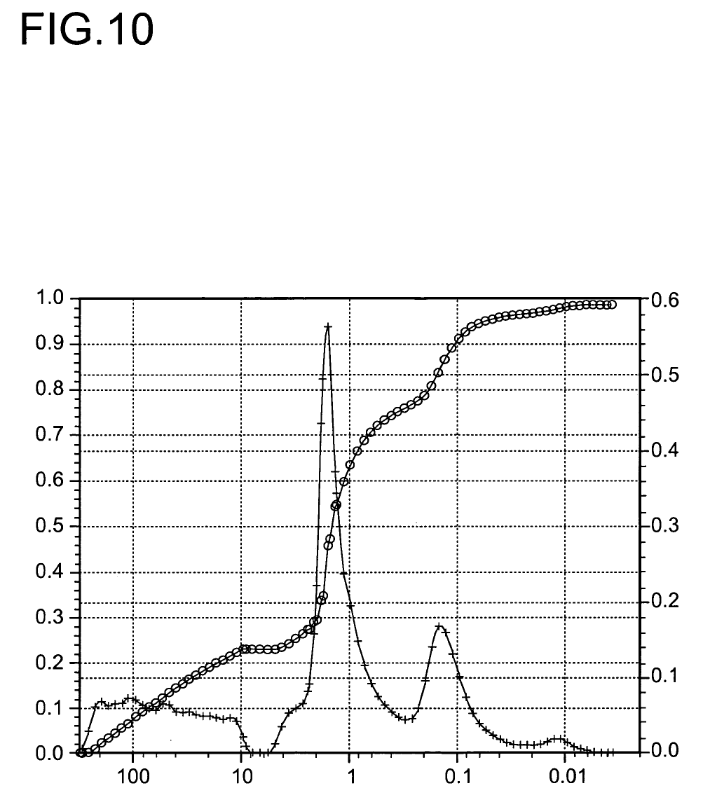

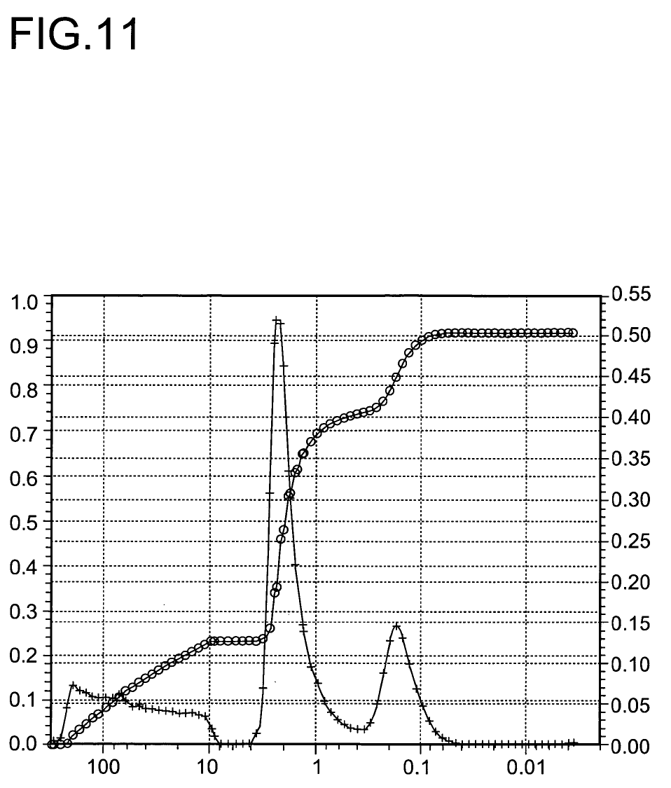

FIG. 10 shows the result of the accompanying mercury porosimetry investigation. The abscissa shows the pore diameter in μm (logarithmic plot). The right ordinate (o curve) shows the integral over the contributions of the individual pore diameters to the total pore volume in ml/g. The left ordinate (+curve) shows the logarithm of the contribution of the individual pore diameter to the total pore volume in ml/g. FIG. 11 shows the corresponding investigation result for the ground rings before they were washed with nitric acid.

On completion of grinding, this active composition, like the active composition of CCM1, was shaped to an annular coated catalyst CCP5.

H) Preparation of an Annular Coated Catalyst CCP6 for the Postreaction Stage Having the Stoichiometry

(Mo₁₂V₃W_{1.2}Cu_{2.4}Oₓ)_{9 parts by weight}(Mo₁V_{0.29}Te_{0.14}Nb_{0.13}Oₓ)_{1 part by weight}

The preparation was as for the preparation of CCM1, but the shaping was effected using a homogeneous mixture (mixed homogeneously on a roller table for 2 h) which consisted of 90% by weight of the active composition powder which was used to prepare CCM1 and 10% by weight of the active composition powder which was used to prepare CCP5.

I) Preparation of an Annular Coated Catalyst CCM2 for the Second Reaction Stage of the Main Reaction (Acrolein→Acrylic Acid) Having the Stoichiometry
(Mo₁₂V_{3.46}W_{1.39})_{0.87}(CuMo_{0.5}O₄)_{0.4}(CuSb₂O₆)_{0.4} as the active composition
1. Preparation of a Starting Composition 1 (Phase 1) Having the Stoichiometry Cu₁Mo_{0.5}W_{0.5}O₄

98 l of a 25% by weight aqueous NH₃ solution were added to 603 l of water. Subsequently, 100 kg of copper(II) acetate hydrate (content: 40.0% by weight of CuO) were dissolved in the aqueous mixture to give a clear, deep blue aqueous solution 1 which contained 3.9% by weight of Cu and was at room temperature.

Independently of solution 1, 620 l of water were heated to 40° C. 27.4 kg of ammonium heptamolybdate tetrahydrate (81.5% by weight of MoO₃) were dissolved therein with stirring within 20 min while maintaining the 40° C. 40.4 kg of ammonium paratungstate heptahydrate (88.9% by weight of WO₃) were then added and, after heating to 90° C., dissolved with stirring at this temperature within 45 min. A clear, yellow/orange-colored aqueous solution 2 was obtained.

Subsequently, the aqueous solution 1 was stirred into solution 2 at 90° C., in the course of which the temperature of the overall mixture did not fall below 80° C. The resulting aqueous suspension was stirred at 80° C. for 30 min. Afterward, it was spray-dried using an S-50-N/R spray dryer from Niro-Atomizer (Copenhagen) (gas inlet temperature: 315° C., gas outlet temperature: 110° C., cocurrent). The spray powder had a particle diameter of from 2 to 50 μm.

100 kg of light green spray powder obtained in this way were metered into a VIU 160 kneader (Sigma blades) from AMK (Aachener Misch-und Knetmaschinen Fabrik) and kneaded with the addition of 8 l of water (residence time: 30 min, temperature: 40 to 50° C.). Afterward, the kneaded material was emptied into an extruder and shaped to extrudates (length: 1-10 cm; diameter: 6 mm) by means of the extruder (from Bonnot Company (Ohio), type: G 103-10/D7A-572K (6″ Extruder W/Packer)). On a belt dryer, the extrudates were dried at a temperature of 120° C. (material temperature) for 1 h. The dried extrudates were subsequently thermally treated (calcined) in the rotary tube furnace described under “1.” as follows:
the thermal treatment was effected continuously with a material input of 50 kg/h of extrudates;
the inclination angle of the rotary tube to the horizontal was 2°;
in countercurrent to the material, an air stream of 75 m³ (STP)/h was conducted through the rotary tube and was supplemented by a total of (2×25) 50 m³ (STP)/h of barrier gas at 25° C.;
the pressure downstream of the rotary tube outlet was 0.8 mbar below the external pressure;
the rotary tube rotated to the left at 1.5 revolutions/min;
no cycle gas method was employed;
in the first pass of the extrudates through the rotary tube, the temperature of the external rotary tube wall was set to 340° C.; the air stream was conducted into the rotary tube at a temperature of from 20 to 30° C.;
subsequently, the extrudates were conducted through the rotary tube at the same throughput rate and under the same conditions, apart from the following differences:
   the temperature of the rotary tube wall was set to 790° C.;
   the air stream was conducted into the rotary tube heated to a temperature of 400° C.

Subsequently, the extrudates having a red-brown color were ground to an average particle diameter of from 3 to 5 μm on a BQ 500 Biplex crossflow classifying mill from Hosokawa-Alpine (Augsburg). The starting composition 1 obtained in this way had a BET surface area of ≦1 m²/g. X-ray diffraction was used to determine the following phases:
1. CuMoO₄-III having wolframite structure;
2. HT copper molybdate.
   2. Preparation of the Starting Composition 2 (Phase 2) Having the Stoichiometry CuSb₂O₆

52 kg of antimony trioxide (99.9% by weight of Sb₂O₃, senarmonite) were suspended with stirring in 216 l of water (25° C.). The resulting aqueous suspension was heated to 80° C. Subsequently, stirring was continued while maintaining the 80° C. for 20 min. Subsequently, 40 kg of a 30% by weight aqueous hydrogen peroxide solution were added within one hour, in the course of which the temperature of 80° C. was maintained. While maintaining this temperature, stirring was continued for 1.5 hours. 20 l of water at 60° C. were then added to obtain an aqueous suspension 1. 618.3 kg of an aqueous ammoniacal copper(II) acetate solution (60.8 g of copper acetate per kg of solution and 75 l of a 25% by weight aqueous ammonia solution in the 618.3 kg of solution) were stirred into this solution at a temperature of 70° C. The mixture was then heated to 95° C. and stirring was continued at this temperature for 30 min. Another 50 l of water at 70° C. were then added and the mixture was heated to 80° C.

Finally, the aqueous suspension was spray-dried (S-50-N/R spray dryer from Niro-Atomizer (Copenhagen), gas inlet temperature 360° C., gas outlet temperature 110° C., cocurrent). The spray powder had a particle diameter of from 2 to 50 μm. 75 kg of spray powder obtained in this way were metered into a VIU 160 kneader (Sigma blades) from AMK (Aachener Misch-und Knetmaschinen Fabrik) and kneaded with the addition of 12 l of water (residence time: 30 min, temperature from 40 to 50° C.). Afterward, the kneaded material was emptied into an extruder (same extruder as in phase 1 preparation) and shaped to extrudates (length 1-1 0 cm; diameter 6 mm) using the extruder. On a belt dryer, the extrudates were dried at a temperature of 120° C. (material temperature) for 1 h.

250 kg of extrudates obtained in this way were thermally treated (calcined) in the rotary tube furnace according to FIG. 1 (described in detail in “B) 1.”) as follows:
the thermal treatment was effected batchwise with a material amount of 250 kg;
the inclination angle of the rotary tube to the horizontal was ≈0°;
the rotary tube rotated to the right at 1.5 revolutions/min;
a gas stream of 205 m³ (STP)/h was conducted through the rotary tube; at the start of the thermal treatment, this consisted of 180 m³ (STP)/h of air and 1×25 m³ (STP)/h of N₂ as barrier gas; the gas stream leaving the rotary tube was supplemented by a further 1×25 m³ (STP)/h of N₂; of this overall stream, 22-25% by volume was recycled into the rotary tube and the remainder discharged; the discharge amount was supplemented by the barrier gas and the remainder by fresh air;
the gas stream was conducted into the rotary tube at 25° C.;
the pressure downstream of the rotary tube outlet was 0.5 mbar below external pressure (atmospheric pressure);
the temperature in the material was initially increased linearly from 25° C. to 250° C. within: 1.5 h; the temperature in the material was then increased linearly from 250° C. to 300° C. within. 2 h and this temperature was maintained for 2 h; then the temperature in the material was increased linearly from 300° C. to 405° C. with in 3 h and this temperature was subsequently maintained for 2 h; the heating zones were then switched off and the temperature within the material was reduced to a temperature below 100° C. within 1 h and finally to ambient temperature by activating the rapid cooling of the rotary tube by aspirating air.

The thus resulting pulverulent starting composition 2 had a specific BET surface area of 0.6 m²/g and the composition CuSb₂O₆. The powder x-ray diagram of the resulting powder showed substantially the reflections of CuSb₂O6 (comparative spectrum 17-0284 of the JCPDS-ICDD index).

3. Preparation of the Starting Composition 3 Having the Stoichiometry Mo₁₂V_{3.35}W_{1.38}

A stirred tank was initially charged with 900 l of water at 25° C. with stirring. Subsequently, 122.4 kg of ammonium heptamolybdate tetrahydrate (81.5% by weight of MoO₃) were added and the mixture was heated to 90° C. with stirring. Initially 22.7 kg of ammonium metavanadate and finally 20.0 kg of ammonium paratungstate heptahydrate (88.9% by weight of WO₃) were then stirred in while maintaining the 90° C. Stirring at 90° C. for a total of 80 minutes resulted in a clear orange-colored solution. This was cooled to 80° C. While maintaining the 80° C., initially 18.8 kg of acetic acid (≈100% by weight, glacial acetic acid) and then 24 l of 25% by weight aqueous ammonia solution were then stirred in.

The solution remained clear and orange-colored and was spray-dried using an S-50-N/R spray dryer from Niro-Atomizer (Copenhagen) (gas inlet temperature: 260° C., gas outlet temperature: 110° C., cocurrent). The resulting spray powder formed the starting composition 3 and had a particle diameter of from 2 to 50 μm.

4. Preparation of the Dry Composition to be Treated Thermally and Having the Stoichiometry (Mo₁₂V_{3.46}W_{1.39})_{0.87} (CuMo_{0.5}W_{0.5}O₄)_{0.4} (CuSb₂O₆)_{0.4}

In a VIU 160 trough kneader having two Sigma blades from AMK (Aachener Misch-und Knetmaschinen Fabrik), 75 kg of starting composition 3, 5.2 l of water and 6.9 kg of acetic acid (100% by weight glacial acetic acid) were initially charged and kneaded over 22 min. Subsequently, 3.1 kg of starting composition 1 and 4.7 kg of starting composition 2 were added and kneaded over a further 8 min (T≈40 to 50° C.).

Afterward, the kneaded material was emptied into an extruder (same extruder as in phase 1 preparation) and shaped to extrudates (length from 1 to 10 cm, diameter 6 mm) by means of the extruder. These were then dried at a temperature (material temperature) of 120° C. over 1 h on a belt dryer.

306 kg of the dried extrudates were subsequently thermally treated in the rotary tube furnace according to FIG. 1 (described in more detail in “B) 1.”) as follows:
the thermal treatment was effected batchwise with a material amount of 306 kg;
the inclination angle of the rotary tube to the horizontal was ≈0°;
the rotary tube rotated to the right at 1.5 revolutions/min;
initially, the material temperature was increased substantially linearly from 25° C. to 100° C. within 2 h;
   during this time, a (substantially) nitrogen stream of 205 m³ (STP)/h is fed through the rotary tube. In the steady state (after displacement of air which was originally present), this has the following composition:
      110 m³ (STP)/h of baseload nitrogen (20),
      25 m³ (STP)/h of barrier gas nitrogen (11), and
      70 m³ (STP)/h of recirculated cycle gas (19).

The barrier gas nitrogen was fed at a temperature of 25° C. The mixture of the two other nitrogen streams was conducted into the rotary tube in each case at the temperature that the material had in each case in the rotary tube.
subsequently, the material temperature was increased from 100° C. to 320° C. at a heating rate of 0.7° C./min;
   until a material temperature of 300° C. had been attained, a gas stream of 205 m³ (STP)/h was conducted through the rotary tube and had the following composition:
      110 m³ (STP)/h consisting of baseload nitrogen (20) and gases released in the rotary tube,
      25 m³ (STP)/h of barrier gas nitrogen (11) and
      70 m³ (STP)/h of recirculated cycle gas (19).

The barrier gas nitrogen was fed at a temperature of 25° C. The mixture of the two other gas streams was conducted into the rotary tube in each case at the temperature that the material had in each case in the rotary tube.

From the exceedance of the material temperature of 160° C. to the attainment of a material temperature of 300° C., 40 mol % of the total amount of ammonia MA released in the course of the overall thermal treatment of the material was released from the material.
on attainment of the material temperature of 320° C., the oxygen content of the gas stream fed to the rotary tube was increased from 0% by volume to 1.5% by volume and maintained over the following 4 h.

At the same time, the temperature in the four heating zones heating the rotary tube was reduced by 5° C. (to 325° C.) and maintained thus over the following 4 h.

The material temperature passed through a temperature maximum above 3250C which did not exceed 340° C. before the material temperature fell back to 325° C.

The composition of the gas stream conducted through the rotary tube of 205 m³ (STP)/h was changed as follows over this period of 4 h:
95 m³ (STP)/h consisting of baseload nitrogen (20) and gases released in the rotary tube;
25 m³ (STP)/h of barrier gas nitrogen (11);
70 m³ (STP)/h of recirculated cycle gas; and
15 m³ (STP)/h of air via the splitter (21).

The barrier gas nitrogen was fed at a temperature of 25° C.

The mixture of the other gas streams was in each case conducted into the rotary tube at the temperature that the material had in each case in the rotary tube.

From the exceedance of the material temperature of 300° C. until the 4 h had elapsed, 55 mol % of the total amount of ammonia M^{A} released in the course of the overall thermal treatment of the material was released from the material (a total of 40 mol %+55 mol %=95 mol % of the amount of ammonia MA had thus been released before the 4 h had elapsed).
when the 4 h had elapsed, the temperature of the material was increased to 400° C. within about 1.5 h at a heating rate of 0.85° C./min.

Subsequently, this temperature was maintained for 30 min.

The composition of the gas stream fed to the rotary tube of 205 m³ (STP)/h over this time was as follows:
95 m³ (STP)/h composed of baseload nitrogen (20) and gases released in the rotary tube;
15 m³ (STP)/h of air (splitter (21));
25 m³ (STP)/h of barrier gas nitrogen (11); and
70 m³ (STP)/h of recirculated cycle gas.

The barrier gas nitrogen was fed at a temperature of 25° C. The mixture of the other gas streams was in each case conducted into the rotary tube at the temperature that the material had in each case in the rotary tube.
reducing the temperature of the material ended the calcination; to this end, the heating zones were switched off and the rapid cooling of the rotary tube by aspirating air was switched on, and the material temperature was reduced to a temperature below 100° C. within 2 h and finally to ambient temperature;
with the switching-off of the heating zones, the composition of the gas stream fed to the rotary tube of 205 m³ (STP)/h was changed to the following mixture:
   110 m³ (STP)/h composed of baseload nitrogen (20) and gases released in the rotary tube;
   0 m³ (STP)/h of air (splitter (21));
   25 m³ (STP)/h of barrier gas nitrogen (11); and
   70 m³ (STP)/h of recirculated cycle gas.

The gas stream was fed to the rotary tube at a temperature of 25° C.
over the entire thermal treatment, the pressure (immediately) downstream of the rotary tube outlet was 0.2 mbar below the external pressure.

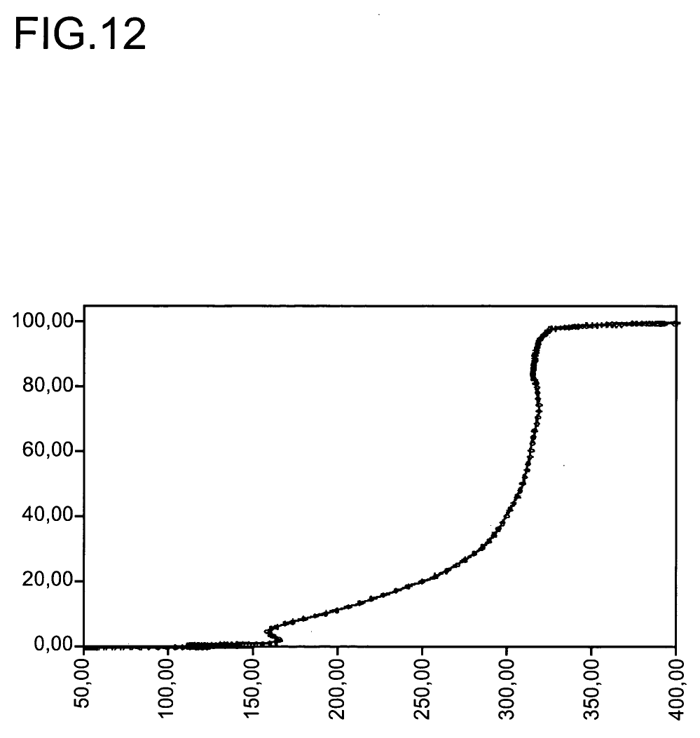

FIG. 12 shows the percentage of M^{A} as a function of the material temperature in ° C.

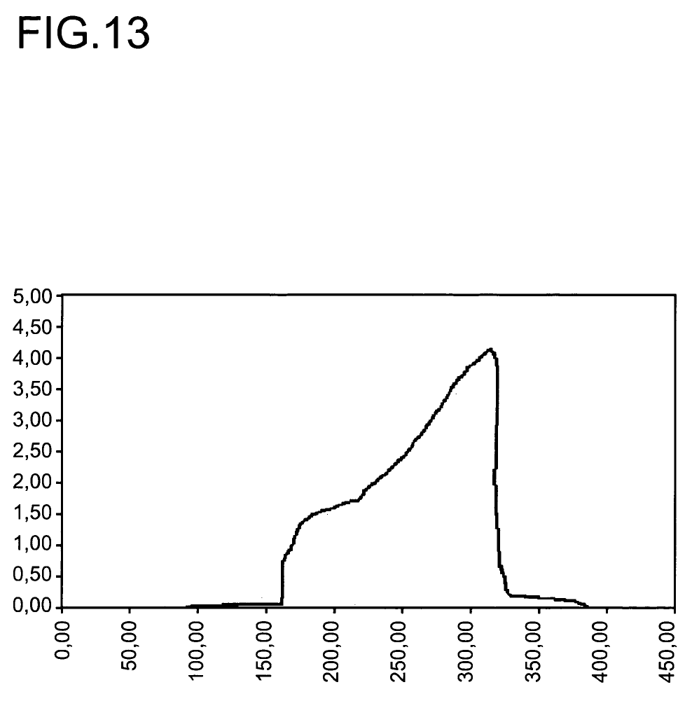

FIG. 13 shows the ammonia concentration of the atmosphere A in % by volume over the thermal treatment as a function of the material temperature in ° C.

6. Shaping of the Multimetal Oxide Active Composition

The catalytically active material obtained under “5.” was ground by means of a BQ500 Biplex crossflow classifying mill (Hosokawa-Alpine Augsburg) to give a fine powder of which 50% of the powder particles passed through a sieve of mesh width from 1 to 10 μm and whose proportion of particles of longest dimension above 50 μm was less than 1%.

70 kg of annular support bodies (external diameter 7.1 mm, length 3.2 mm, internal diameter 4.0 mm; type C220 steatite from CeramTec having a surface roughness R_{z} of 45 μm and a total pore volume based on the volume of the support body of ≦1% by volume; cf. DE-A 2135620) were charged into a coating tank (inclination angle 90°; Hicoater from Lödige, Germany) of capacity 200 l. Subsequently, the coating tank was set into rotation at 16 rpm. A nozzle was used to spray from 3.8 to 4.2 liters of an aqueous solution composed of 75% by weight of water and 25% by weight of glycerol onto the support bodies within 25 min. At the same time, 18.1 kg of the ground multimetal oxide active composition were metered in continuously within the same period via an agitated channel outside the spray cone of the atomizer nozzle. During the coating, the powder supplied was fully taken up on the surface of the support body; no agglomeration of the finely divided oxidic active composition was observed. On completion of addition of active composition powder and water, hot air (approx. 400 m³/h) at 100° C. (alternatively from 80 to 120° C.) was blown into the coating tank at a rotation rate of 2 rpm for 40 min (alternatively from 15 to 60 min). Annular coated catalysts CCM2 were obtained whose proportion of oxidic active composition based on the overall composition was 20% by weight. The coating thickness, viewed both over the surface of one support body and over the surface of different support bodies, was 170±50 μm.

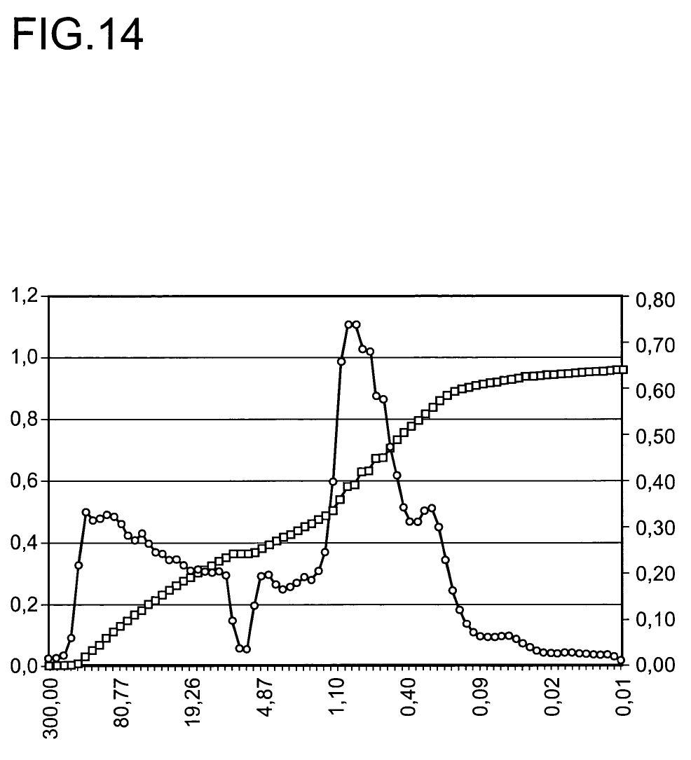

FIG. 14 also shows the pore distribution of the ground active composition powder before it is shaped (its specific BET surface area was 21 m²/g). On the abscissa is plotted the pore diameter in μm (logarithmic scale).

On the right ordinate is plotted the logarithm of the differential contribution in ml/g of the particular pore diameter to the total pore volume (O curve). The maximum indicates the pore diameter having the greatest contribution to the total pore volume. On the left ordinate is plotted in ml/g the integral over the individual contributions of the individual pore diameters to the total pore volume (l curve). The end point is the total pore volume (unless stated otherwise, all data in this document on determinations of total pore volumes and on diameter distributions to these total pore volumes relate to determinations by the method of mercury porosimetry using the Auto Pore 9220 instrument from Micromeritics GmbH, 4040 Neuss, Germany (bandwidth from 30 Å to 0.3 mm); all data in this document on determinations of specific surface areas or of micropore volumes relate to determinations to DIN 66131 (determination of the specific surface area of solids by Brunauer-Emmet-Teller (BET) gas adsorption (N₂))).

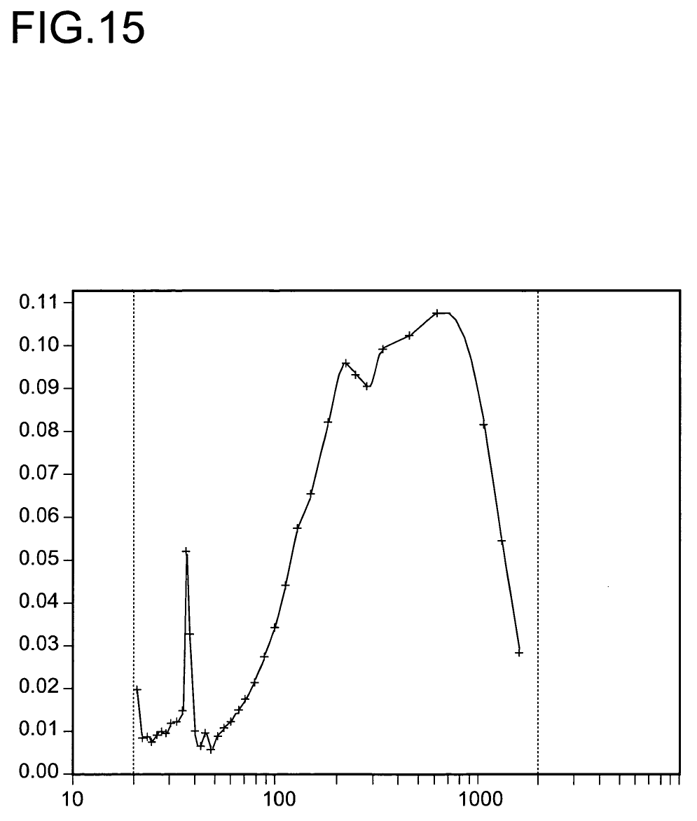

FIG. 15 shows, for the active composition powder before it is shaped, in ml/q (ordinate), the individual contributions of the individual pore diameters (abscissa, in angstrøm, logarithmic scale) in the micropore range to the total pore volume.

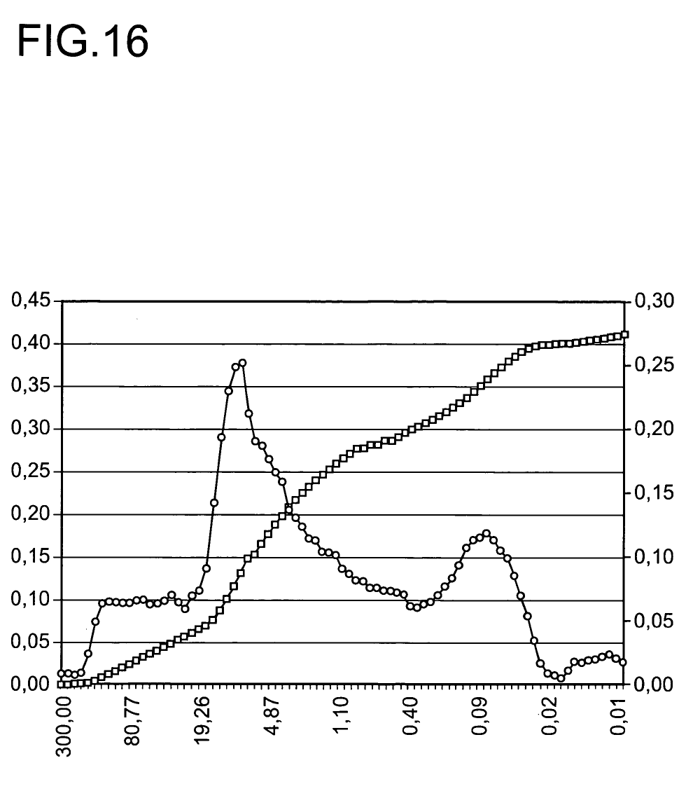

FIG. 16 shows the same as FIG. 14, but for multimetal oxide active composition subsequently removed from the annular coated catalyst by mechanical scraping (its specific surface area was 24.8 m²/g).

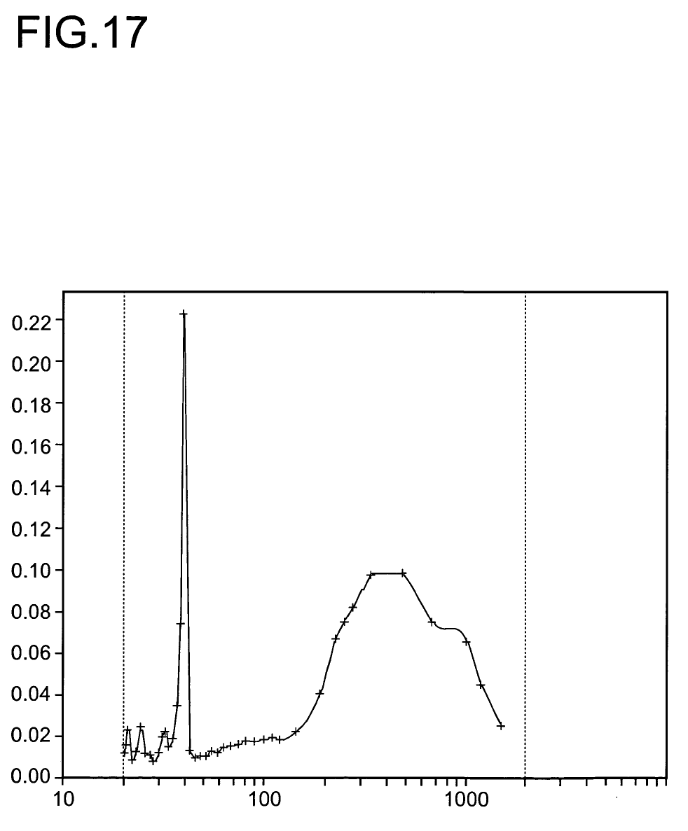

FIG. 17 shows the same as FIG. 15, but for multimetal oxide active composition subsequently remove from the annular coated catalyst by mechanical scraping.

Doping as described for the coated catalysts CCP1 to CCP6 (for example with Pd or Fe or Cu content increased) and/or blending allows, instead of CCM2, coated catalysts to be obtained for an inventive postreaction stage.

K) Description of the Experimental Arrangement for the Two Reaction Stages of the Main Reaction and for the Postreaction Stage, and Reaction Description

First Reaction Stage of the Main Reaction

A reaction tube (V2A steel; external diameter 30 mm, wall thickness 2.5 mm, internal diameter 25 mm, length: 320 cm) was charged from top to bottom as follows:

| | |
|---|---|
| Section 1: | length 50 cm |
| | Steatite rings of geometry 7 mm × 7 mm × 4 mm (external |
| | diameter × length × internal diameter) as a preliminary bed. |
| Section 2: | length 100 cm |
| | Catalyst charge with a homogeneous mixture of steatite rings |
| | of geometry 5 mm × 3 mm × 2 mm (external diameter × |
| | length × internal diameter) and 70% by weight of the annular |
| | unsupported catalyst UCM. |
| Section 3: | length 170 cm |
| | Catalyst charge with exclusively annular unsupported catalyst |
| | UCM. |

The reaction tube was thermostatted in countercurrent using a nitrogen-sparged salt bath (53% by weight potassium nitrate, 40% by weight sodium nitrite and 7% by weight sodium nitrate).

Second Reaction Stage of the Main Reaction

A reaction tube (V2A steel; external diameter 30 mm, wall thickness 2.5 mm, internal diameter 25 mm, length: 320 cm) was charged from top to bottom as follows:

| | |
|---|---|
| Section 1: | length 20 cm |
| | Preliminary bed of steatite rings of geometry 7 mm × 7 mm × |
| | 4 mm (external diameter × length × internal diameter). |
| Section 2: | length 100 cm |
| | Catalyst charge with a homogeneous mixture of 25% by |
| | weight of steatite rings of geometry 7 mm × 3 mm × 4 mm |
| | (external diameter × length × internal diameter) and 75% by |
| | weight of the annular coated catalyst CCM1 (alternatively |
| | CCM2 may also be used here). |
| Section 3: | length 200 cm |
| | Catalyst charge with exclusively annular coated catalyst |
| | CCM1 (alternatively CCM2 may also be used here). |

The reaction tube was thermostatted in countercurrent by means of a nitrogen-sparged salt bath (53% by weight potassium nitrate, 40% by weight sodium nitrite and 7% by weight sodium nitrate).

Between the two reaction stages was disposed an intermediate cooler charged with inert material, through which the product gas mixture of the first reaction stage was conducted and was cooled to a temperature of 250° C. by indirect cooling.

The first reaction stage was charged continuously with a starting reaction gas mixture of the following composition:
from 6 to 6.2% by volume of propene (polymer-grade),
from 3 to 3.2% by vol. of H₂O,
from 0.3 to 0.5% by vol. of CO,
from 0.9 to 1.1% by vol. of CO₂,
from 0.01 to 0.02% by vol. of acrolein,
from 10.4 to 10.6% by vol. of O₂ and,
   as the remainder ad 100% by vol. of molecular nitrogen.

The hourly space velocity on the catalyst charge with propene selected was 100 l (STP)/l/h. The salt bath temperature of the first reaction stage was 338° C. The salt bath temperature of the second reaction stage was 264° C. To the product gas mixture leaving the intermediate cooler at a temperature of 250° C. was metered sufficient compressed air which had a temperature of 140° C. that a starting reaction gas mixture was fed to the second reaction stage in which the ratio of molecular oxygen to acrolein (ratio of the percentages by volume) was 1.3.

Gas chromatography analysis at the outlet of the second reaction stage gave the following results (the condensate of the gas mixture leaving the second reaction stage was analyzed; all constituents whose boiling point (1 atm) is ≧−50° C. were condensed):
Propene conversion: 97.5 mol %;
selectivity of acrylic acid formation (S^{AA}ₚₑₙ): 91.7 mol %;
overall selectivity of secondary component formation (Sₒᵥₑ): 1.7 mol %.

In addition, the following contents were present at the outlet of the second reaction stage:
acrolein: 500 ppm by volume;
acetaldehyde: 0.03% by volume;
acetic acid: 0.14% by volume.
   Postreaction Stage

A reaction tube (V2A steel; external diameter 30 mm, wall thickness 2.5 mm, internal diameter 25 mm, length 320 cm) was in each case charged over its entire length with one of the annular coated catalysts CCP1 to CCP6. The reaction tube was thermostafted in countercurrent by means of a nitrogen-sparged salt bath (53% by weight potassium nitrate, 40% by weight sodium nitrite and 7% by weight sodium nitrate).

The reaction gas mixture leaving the second reaction stage was conducted directly into the postreaction stage without intermediate air feeding and without intermediate cooling. The reaction gas mixture leaving the postreaction tube was (in a manner corresponding to that already described for the reaction gas mixture leaving the second reaction stage of the main reaction) analyzed by gas chromatography. As a function of the selected postreactor charge and salt bath temperature, the following results were obtained:

CCP1, salt bath temperature=270° C.:

| | |
|---|---|
| Propene conversion: | 97.5 mol %; |
| S^{AA}ₚₑₙ: | 90.8 mol %; |
| Sₒᵥₑ: | 0.85 mol %; |
| Acrolein: | 90 ppm by volume; |
| Acetaldehyde: | 0.02% by volume; |
| Acetic acid: | 0.07% by volume. |

CCP2, salt bath temperature=250° C.:

| | |
|---|---|
| Propene conversion: | 97.5 mol %; |
| S^{AA}ₚₑₙ: | 90.7 mol %; |
| Sₒᵥₑ: | 0.83 mol %; |
| Acrolein: | 41 ppm by volume; |
| Acetaldehyde: | 0.01% by volume; |
| Acetic acid: | 0.15% by volume. |

CCP3, salt bath temperature=250° C.:

| | |
|---|---|
| Propene conversion: | 97.5 mol %; |
| S^{AA}ₚₑₙ: | 91.7 mol %; |
| Sₒᵥₑ: | 0.74 mol %; |
| Acrolein: | 20 ppm by volume; |
| Acetaldehyde: | 0.01% by volume; |
| Acetic acid: | 0.17% by volume. |

CCP4, salt bath temperature=250° C.:

| | |
|---|---|
| Propene conversion: | 97.5 mol %; |
| S^{AA}ₚₑₙ: | 91.7 mol %; |
| Sₒᵥₑ: | 0.69 mol %; |
| Acrolein: | 30 ppm by volume; |
| Acetaldehyde: | <0.01% by volume; |
| Acetic acid: | 0.15% by volume. |

CCP5, salt bath temperature=270° C.:

| | |
|---|---|
| Propene conversion: | 98.5 mol %; |
| S^{AA}ₚₑₙ: | 92.2 mol %; |
| Sₒᵥₑ: | 0.78 mol %; |
| Acrolein: | 80 ppm by volume; |
| Acetaldehyde: | <0.01% by volume; |
| Acetic acid: | 0.19% by volume. |

CCP6, salt bath temperature=230° C.:

| | |
|---|---|
| Propene conversion: | 97.7 mol %; |
| S^{AA}ₚₑₙ: | 91.7 mol %; |
| Sₒᵥₑ: | 0.83 mol %; |
| Acrolein: | 30 ppm by volume; |
| Acetaldehyde: | <0.01% by volume; |
| Acetic acid: | 0.16% by volume. |

Instead of being charged with one of the annular coated catalysts CCP1 to CCP6, the postreaction stage was charged in further experiments with one of the following coated catalysts and the following results were obtained: CCP7:
Active composition stoichiometry: Mo₁₂V₃W_{1.2}Cu_{2.4}Bi_{1.0}Oₓ

The preparation was as for the preparation of CCP1, but with the difference that, after solution 1 had been stirred into solution 2, 17.0 kg of solid Bi(NO₃)₃.5H₂O (manufacturer: ABCR, 98%) were stirred into the resulting mixture before the aqueous NH₃ solution was added.

Result:

Salt bath temperature=250° C.
Propene conversion: 97.5 mol %
Sₚₑₙ^{AA}: 91.8 mol %
Sₒᵥₑ: 0.69 mol %
Acrolein: 20 ppm by volume;
Acetaldehyde: <0.01% by volume;
Acetic acid: 0.16% by volume.
   CCP8:
   Active Composition Stoichiometry: Mo₁₂V₃W_{1.2}Cu_{2.4}Co_{1.0}Oₓ

The preparation was as for the preparation of CCP1, but with the difference that, after solution 1 had been stirred into solution 2, 10.5 kg of solid Co(NO₃)₂.6H₂O (manufacturer: ABCR, 98%) were stirred into the resulting mixture before the aqueous NH₃ solution was added.

Result:

Salt bath temperature=250° C.
Propene conversion: 97.5 mol %
Sₚₑₙ^{AA}: 91.7 mol %
Sₒᵥₑ: 0.70 mol %
Acrolein: 24 ppm by volume;
Acetaldehyde: 0.01% by volume;
Acetic acid: 0.16% by volume.

U.S. Provisional Patent Application No. 60/566,398, filed on April, 30, 2004, is incorporated into the present application by literature reference. With regard to the above-mentioned teachings, numerous alterations and deviations from the present invention are possible. It can therefore be assumed that, within the scope of the appended claims, the invention may be performed differently to the way specifically described herein.

1. A process for preparing acrylic acid by heterogeneously catalyzed partial oxidation of at least one C₃ hydrocarbon precursor compound, wherein the overall selectivity of secondary component formation Sₒᵥₑ is ≦1.5 mol %.

2. The process according to claim 1, wherein Sₒᵥₑ is ≦1.3 mol %.

3. The process according to claim 1, wherein Sₒᵥₑ is ≦1.0 mol %.

4. The process according to claim 1, wherein Sₒᵥₑ is ≦0.8 mol %.

5. The process according to any of claims 1 to 4, wherein the C₃ hydrocarbon precursor compound is propene and the propene conversion C^{Pen} is ≧95 mol %.

6. The process according to any of claims 1 to 4, wherein the C₃ hydrocarbon precursor compound is propene and the propene conversion C^{Pen} is ≧96 mol %.

7. The process according to any of claims 1 to 4, wherein the C₃ hydrocarbon precursor compound is propene and the propene conversion C^{Pen} is ≧97 mol %.

8. The process according to any of claims 1 to 4, wherein the C₃ hydrocarbon compound is propene and the propene conversion C^{Pen} is ≧98 mol %.

9. The process according to any of claims 1 to 8, wherein the C₃ hydrocarbon compound is propene, and the selectivity of acrylic acid formation S^{AA}ₚₑₙ is ≧90 mol %.

10. The process according to any of claims 1 to 8, wherein the C₃ hydrocarbon compound is propene, and the selectivity of acrylic acid formation S^{AA}ₚₑₙ is ≧92 mol %.

11. The process according to any of claims 1 to 8, wherein the C₃ hydrocarbon compound is propene, and the selectivity of acrylic acid formation S^{AA}ₚₑₙ is ≧94 mol %.

12. The process according to any of claims 1 to 11, wherein acrylic acid is initially obtained in a main reaction by heterogeneously catalyzed partial oxidation of at least one C₃ hydrocarbon precursor compound in such a way that the overall selectivity of secondary component formation Sₒᵥₑ is ≧1.7 mol % and the resulting product gas mixture is subsequently, if appropriate after addition of inert gas or of molecular oxygen or of a mixture of molecular oxygen and inert gas, in a postreaction stage at elevated temperature, conducted through a catalyst charge in such a way that the acrylic acid present in the product gas mixture remains substantially unchanged, while the secondary components present in the product gas mixture are at least partly combusted to carbon oxides and water.

13. The process according to claim 12, wherein the C₃ hydrocarbon precursor compound is propane.

14. The process according to claim 12, wherein the C₃ hydrocarbon precursor compound is propene.

15. The process according to claim 14, wherein the propene conversion C^{pen} in the main reaction is ≧95 mol %.

16. The process according to claim 14, wherein the propene conversion C^{pen} in the main reaction is ≧96 mol %.

17. The process according to any of claims 14 to 16, wherein the selectivity of acrylic acid formation S^{AA}ₚₑₙ in the main reaction is ≧90 mol %.

18. The process according to any of claims 14 to 16, wherein the selectivity of acrylic acid formation S^{AA}ₚₑₙ in the main reaction is ≧95 mol %.

19. The process according to any of claims 14 to 18, wherein acrylic acid is obtained in the main reaction by a two-stage heterogeneously catalyzed partial oxidation of propene, and both the hourly space velocity on the catalyst bed of the first reaction stage with propene and the hourly space velocity on the catalyst bed of the second reaction stage with acrolein are in the range from a 120 l (STP)/l/h and ≦300 l (STP)/l/h.

20. The process according to claim 19, wherein both the hourly space velocity on the catalyst bed of the first reaction stage with propene and the hourly space velocity on the catalyst bed of the second reaction stage with acrolein are in the range from ≧130 l (STP)/l/h and ≦300 l (STP)/l/h.

21. The process according to any of claims 12 to 20, wherein the active composition of the catalyst charge of the postreaction stage is at least one multimetal oxide of the general formula I
Mo₁₂VₐX¹_{b}X²_{c}X³_{d}X⁴ₑX⁵_{f}X⁶_{g}X⁷ₕOₙ (I)
where
X¹=W, Nb, Ta, Cr and/or Ce,
X²=Cu, Ni, Co, Fe, Mn and/or Zn,
X³=Sb and/or Bi,
X⁴=one or more alkali metals,
X⁵=one or more alkaline earth metals,
X⁶=Si, Al, Ti and/or Zr,
X⁷=Pd, Pt, Ag, Rh and/or Ir,
a=from 1 to 6,
b=from 0.2 to 4,
c=from 0.5 to 18,
d=from 0 to 40,
e=from 0 to 2,
f=from 0 to 4,
g=from 0 to 40,
h=from o to 1, and
n=a number which is determined by the valency and frequency of the elements in I other than oxygen.

22. The process according to any of claims 12 to 20, wherein the active composition of the catalyst charge of the postreaction stage is at least one multimetal oxide of the general formula III
Mo₁VₐM¹_{b}M²_{c}M³_{d}Oₙ (III)
where
M¹=at least one of the elements from the group consisting of Te and Sb;
M²=at least one of the elements from the group consisting of Nb, Ti, W, Ta and Ce;
M³=at least one of the elements from the group consisting of Pb, Ni, Co, Bi, Pd, Ag, Pt, Cu, Au, Ga, Zn, Sn, In, Re, Ir, Sm, Sc, Y, Pr, Nd and Tb;
a=from 0.01 to 1,
b=from >0 to 1,
c=from >0 to 1,
d=from 0 to 0.5, preferably from >0 to 0.5, and
n=a number which is determined by the valency and frequency of the elements in III other than oxygen,
and an x-ray diffractogram which has reflections h, i and k whose peak locations are at the reflection angles (2⊖) of 22.2±0.5° (h), 27.3±0.5° (i) and 28.2±0.5° (k), and
the reflection h has the highest intensity within the x-ray diffractogram and a half-height width of at most 0.5°,
the intensity Pᵢ of the reflection i and the intensity Pₖ of the reflection k satisfy the relationship 0.20≦R≦0.85 in which R is the intensity ratio defined by the formula
R=Pᵢ/(Pᵢ+Pₖ),
and
the half-height width of the reflection i and of the reflection k are each ≦1°.

23. The process according to any of claims 1 to 4, wherein the C₃ hydrocarbon precursor compound is propane.
